(19) United States
(12) Patent Application Publication
(10) Pub. No.: US 2026/0157626 A1
(43) Pub. Date: Jun. 11, 2026

(54) SYSTEMS AND METHODS FOR VIRTUAL REALITY, AUGMENTED REALITY, AND MIXED REALITY BASED VISUAL FUNCTION ASSESSMENT

(71) Applicant: Apellis Pharmaceuticals, Inc., Waltham, MA (US)

(72) Inventors: Alex S. McKeown, Waltham, MA (US); Lukas Scheibler, Telluride, CO (US); Jason W. Raines, Sudbury, MA (US); Stephan Wyder, Bern (CH); Matthias Pfister, Liebefeld (CH)

(21) Appl. No.: 18/702,594

(22) PCT Filed: Oct. 21, 2022
(86) PCT No.: PCT/US2022/047489

§ 371 Date: Apr. 18, 2024

Related U.S. Application Data
(60) Provisional application No. 63/270,897, filed on Oct. 22, 2021.

(51) Int. Cl. A61B 3/113; A61B 3/00; A61B 3/02; A61B 3/032; A61K 31/015; A61K 31/355; A61K 31/375; A61K 31/7088; A61K 33/30; A61K 33/34; A61K 38/17; A61K 45/06; C07K 16/22; G02B 27/01; G06F 3/01; G06T 7/00; G06T 7/246; G06T 7/62; G06T 7/73

(52) U.S. Cl. CPC A61B 3/113; A61B 3/0025; A61B 3/0058; A61B 3/0091; A61B 3/022; A61B 3/032; A61K 31/015; A61K 31/355; A61K 31/375; A61K 31/7088; A61K 33/30; A61K 33/34; A61K 38/179; A61K 45/06; C07K 16/22; G06F 3/012; G06F 3/013; G06T 7/0016; G06T 7/248; G06T 7/62; G06T 7/74; G02B 27/0172; G06T 2207/30041

(57) ABSTRACT

Presented herein are systems and methods for improved virtual reality (VR), augmented reality (AR), and/or mixed reality (MR)-based visual function assessment. In various embodiments, systems and methods described herein utilize eye tracking to display static and/or changing/moving visual stimuli (targets) to the subject to elicit and record patterns of eye movements relative to the stimuli, where said patterns of eye movements are correlated to disease or retinal dysfunction. In certain embodiments, techniques of functional vision testing automatically perform, in real time, during the course of the functional vision test, a visibility determination to automatically identify if the subject is tracking the target in time and space. In some embodiments, systems and methods align VR headset optics with the wearer's eye and/or render a graphical scotoma mask that simulates the effect of a particular patient's scotoma on a visual field of either the patient or another individual.

### PRIORITY APPLICATION

The present application claims the benefit of U.S. Provisional Patent Application No. 63/270,897, filed on Oct. 22, 2021, the disclosure of which is hereby incorporated by reference in its entirety.

### BACKGROUND

Current eye tracking or gaze estimation systems track eye movements to continuously estimate where on a surface or in a volume of space a subject is looking. Head-mounted eye tracking systems include dedicated camera hardware designed for capturing images of one or both eyes of the subject, and a light source (e.g., an infrared illuminator) to illuminate the eye(s).

Eye tracking systems are predominately used to enhance the player experience in video games by providing an additional element of interactivity. Eye tracking is not as extensively used in optometry or ophthalmology practice for functional evaluation of a patient's vision. Current commercially available eye tracking systems generally lack sensitivity and are poorly correlated to retinal anatomy, as would be necessary for proper clinical evaluation. For example, current virtual reality (VR) headsets measure direction of gaze in a headset reference system, independent of eye anatomy. Moreover, the optical quality of VR headsets is limited due to the compact lens design, resulting in aberrations.

There is a need for improved systems and methods for virtual reality (VR), augmented reality (AR), and/or mixed reality (MR)-based visual function assessment.

### SUMMARY

Presented herein are systems and methods for improved virtual reality (VR), augmented reality (AR), and/or mixed reality (MR)-based visual function assessment. In various embodiments, the systems and methods described herein: (i) utilize an anatomical reference to determine the preferred retinal locus (PRL) of a subject's eye, (ii) align VR headset optics with the wearer's eye, thereby reducing inaccuracy due to aberrations (e.g., chromatic, spheric) near the periphery of VR headset lenses, and/or (iii) render a graphical (e.g., 2D) scotoma mask that simulates the effect of a particular patient's scotoma on a visual field of either the patient (e.g. the patient's good eye) or another individual. Furthermore, in various embodiments, the systems and methods described herein utilize eye tracking with one or more of (i), (ii), and (iii) above to display static and/or changing/moving visual stimuli (targets) to the subject/patient to elicit and record patterns of eye movements relative to the stimuli, where said patterns of eye movements are correlated to disease or retinal dysfunction.

In some embodiments, a functional vision test can be used to diagnose and/or monitor an eye condition, such as geographic atrophy and/or an age-related macular degeneration (AMD) (e.g., wet AMD, neovascular AMD, and/or dry AMD). A functional vision test may provide one or more targets that move and/or change contrast in a virtual and/or augmented space. A subject's point of gaze may be tracked to determine whether it is aligned with one of the targets or not at any given instant. Using time-based parameters, it may be determined whether the subject is tracking a target and/or any target to make a visibility determination. Test results (e.g., of a radial sweep test) may be computed and/or displayed based on the visibility determination. For example, a plot may be generated in a contrast sensitivity-spatial frequency space that is usable to diagnose and/or monitor an eye condition of the subject.

In one aspect, techniques described herein are used to perform a functional vision test, for example utilizing an VR/AR/and/or MR device (e.g., headset) (as used hereinafter, the terms “headset” or “VR headset” refer broadly to head- or face-worn systems that implement virtual reality, augmented reality, and/or mixed reality functionality). A functional vision test may include providing one or more targets (e.g., patches, such as contrast-based patches) in a visual and/or augmented scene (e.g., on a virtual chart in the scene). The target(s) may move over time. A visibility determination may be made to automatically identify if a subject is tracking the one or more targets (e.g., in within the scene. The determination may be based, at least in part, on time-based parameters (e.g., using no other parameters or also using one or more non-time-based parameters). A contrast, spatial position, spatial frequency, and/or direction of movement of the target(s) may change (e.g., decrease) (e.g., abruptly) throughout the test, for example upon determining that the subject has been tracking a target for some amount of time. Test results may be computed and/or displayed using the visibility determination. Test results may include a metric such as an area under curve (AUC) metric that characterizes the subject's vision. A metric may be indicative of presence of, severity of, and/or progression of an eye condition of the subject.

In another aspect, techniques described herein utilize an anatomical reference to determine the preferred retinal locus (PRL) of a subject's eye via a VR/AR/and/or MR headset with eye-tracking capability (as used hereinafter, the terms “headset” or “VR headset” refer broadly to head- or face-worn systems that implement virtual reality, augmented reality, and/or mixed reality functionality). For example, in certain embodiments, two data streams corresponding to gaze direction and gaze origin, respectively, are received. This data may be received from a VR headset system with currently-existing commercial eye tracking capability, where the data produced has a headset reference system, rather than an eye anatomy reference system. The gaze origin data received over time (multiple data points) are used to model a sphere or other geometric volume. A stable reference for the anatomy of the eye—for example, an optical axis of the eye in an anatomy reference system—is then identified—for example, as a line connecting the center of the modeled sphere and the gaze origin. The PRL relative to retina anatomy is then identified using the identified optical axis and the gaze direction.

In yet another aspect, techniques for VR headset adjustment are presented that align headset optics with the eye, thereby reducing inaccuracy due to aberrations (e.g., chromatic, spheric) near the periphery of VR headset lenses. The optical quality of VR headsets is limited due to the compact lens design (e.g., Fresnel lenses, singlets). Proper headset adjustment to vertically (and/or horizontally) align headset optics with the eye enables improved optical quality by reducing or eliminating the effect of aberrations that are generally more prevalent and/or pronounced in the periphery than in the center of the lens. The techniques described herein use eye tracking data to implement virtual iron sights that provide feedback for the user to adjust the headset placement and actively optimize optical alignment.

In yet another aspect, techniques are presented for rendering a graphical (e.g., 2D) scotoma mask that simulates the effect of a particular patient's scotoma on a visual field. The scotoma mask can simulate a scotoma in its current form, or it can simulate the estimated appearance of a scotoma in the future. The techniques described herein use a VR headset with eye tracking capability that provides a point of gaze data stream. Input is received from a patient who suffers from a real scotoma and is used to define the graphical scotoma mask. The graphical scotoma mask is the presented via a VR headset (the same or different headset than used by the patient to create the mask) to an individual (which can be the patient or another individual, such as a relative of the patient) as an overlay to a simulated scene (VR) or real scene (AR). The scotoma mask can be used to improve patient compliance with treatment, e.g., by simulating the effect of the scotoma in the future without treatment), to increase empathy of caring relatives who can experience the way the patient sees with the scotoma via the scotoma mask, and the measure the sensitivity of specific visual function tests during development (e.g., test results of healthy subjects can be compared with and without scotoma simulation).

In yet another aspect, techniques of functional vision testing (e.g., optionally using said changing/moving visual stimuli that benefit from one or more of (i), (ii), and (iii) above) are presented herein that automatically perform, in real time, during the course of the functional vision test, a visibility determination using a machine learning algorithm to automatically identify if the subject is accurately tracking the target in time and space.

In one aspect, the invention is directed to a method for conducting a functional vision test (e.g., a contrast sensitivity and/or spatial frequency test, e.g., a radial sweep test) on a subject using a virtual- and/or augmented- and/or mixed-reality device (e.g., VR headset with eye tracking capability), the method comprising: rendering and displaying (e.g., on a head-mounted display of the VR headset) to the subject, by a processor of a computing device, over a course of the functional vision test, one or more targets (e.g., two or more, three or more, four or more, or five or more targets) within a virtual and/or augmented scene in a visual field of the subject; automatically performing, by the processor, in real time, during the course of the functional vision test, a visibility determination to automatically identify if the subject is tracking the one or more targets (e.g., in time and space) within the virtual and/or augmented scene, wherein the visibility determination is based, at least in part, on time-based parameters; and optionally, computing and/or displaying, by the processor, test results using the visibility determination.

In some embodiments, performing the visibility determination comprises: determining, by the processor, in real time, a point of gaze of the subject; and comparing, by the processor, the point of gaze to a current spatial position of the one or more targets within the virtual and/or augmented scene using the time-based parameters.

In some embodiments, performing the visibility determination comprises: determining, by the processor, in real time, a point of gaze of the subject; and determining, by the processor, whether the point of gaze is aligned with one of the target(s) within the virtual and/or augmented scene. In some embodiments, performing the visibility determination comprises: determining, by the processor, in real time, a point of gaze of the subject; and determining, by the processor, whether the subject is tracking one of the target(s) within the virtual and/or augmented scene using at least one of the time-based parameters. In some embodiments, the time-based parameters each correspond to a time period during which the point of gaze is or is not aligned with one or more of the one or more targets within the virtual and/or augmented scene.

In some embodiments, the method includes automatically adjusting (e.g., decreasing) contrast and/or spatial resolution of one or more of the target(s) within the virtual and/or augmented scene based on the comparison of the point of gaze to the current spatial position of the one or more of the target(s) within the virtual and/or augmented scene (e.g., based on a determination, by the processor, that the point of gaze is aligned to the one or more of the target(s) for a (or at least a) predetermined threshold period of time using one or more of the time-based parameters). In some embodiments, the method includes automatically adjusting contrast, spatial position, spatial resolution, and/or direction of movement of the one or more targets (e.g., abruptly) (e.g., one or more of the one or more targets) within the virtual and/or augmented scene during the test using one or more of the time-based parameters (e.g., making a tracked one of the target(s) lower contrast to increase difficulty, changing direction of movement of a tracked one of the target(s) in an abrupt way to increase difficulty, resetting all of the target(s) with higher contrast, or stopping the test). In some embodiments, adjusting the contrast, spatial position, spatial resolution, and/or direction of movement of the one or more targets comprises: determining, by the processor, in real time, a point of gaze of the subject; and automatically adjusting the contrast and/or spatial resolution of the one or more targets (e.g., of at least one of the one or more targets) within the virtual and/or augmented scene based at least in part on a point of gaze of the subject being aligned with the one or more targets according to at least one of the time-based parameters.

In some embodiments, there are a plurality of targets displayed within the virtual and/or augmented scene in the visual field of the subject, and wherein the time-based parameters comprises (e.g., consists of) four parameters: (i) for tracking when a (e.g., the) point of gaze of the subject is on (e.g., aligned with) a particular target of the plurality of targets within the virtual and/or augmented scene, (ii) for tracking when a (e.g., the) point of gaze of the subject is off (e.g., not aligned with) a particular target of the plurality of targets within the virtual and/or augmented scene, (iii) for tracking when a (e.g., the) point of gaze of the subject is on (e.g., aligned with) any of the plurality of targets within the virtual and/or augmented scene, and (iv) for tracking when a (e.g., the) point of gaze of the subject is off (e.g., not aligned with) all of the plurality of targets within the virtual and/or augmented scene. In some embodiments, the four parameters account for at least 80% (e.g., at least 90%, e.g., at least 95%, e.g., at least 98%, e.g., 100%) of all meaningful variables used in the functional vision test (e.g., where a meaningful variable is a variable that has at least a 5% impact on the outcome of the functional vision test).

In some embodiments, the time-based parameters are asymmetric [e.g., wherein parameter (i) and parameter (ii) refer to (e.g., correspond to) different time lengths and/or wherein parameter (iii) and parameter (iv) refer to (e.g., correspond to) different time lengths (e.g., wherein parameter (i) refers to a shorter time length than parameter (ii) does and/or parameter (iii) refers to a shorter time length than parameter (iv) does].

In some embodiments, (i) at least one of the time-based parameters corresponds to the subject tracking one of the target(s) [e.g., during which a point of gaze is aligned with (e.g., incident on) the one of the target(s) within the virtual and/or augmented scene], (ii) at least one of the time-based parameters corresponds to the subject tracking any of the target(s) [e.g., during which a point of gaze is aligned with (e.g., incident on) any of the target(s) within the virtual and/or augmented scene], or (iii) both (i) and (ii).

In some embodiments, the visibility determination is performed using only the time-based parameters (i.e., no non-time based parameters are used to make the visibility determination). In some embodiments, no more than 10 total parameters (e.g., no more than 8 total parameters, no more than 6 total parameters, no more than 5 total parameters, or no more than 4 total parameters) are used to perform the visibility determination (e.g., and each of the parameters is a time-based parameter).

In some embodiments, the one or more targets move and/or change direction of movement within the virtual and/or augmented scene during the functional vision test [e.g., regardless of whether the subject is or is not tracking the target(s) (e.g., as determined, by the processor, using a point of gaze of the subject)]. In some embodiments, the one or more targets change contrast within the virtual and/or augmented scene during the functional vision test (e.g., based on determining, by the processor, in real time, that the subject is tracking the one or more targets) [e.g., changing contrast and/or spatial resolution of a target only when the subject has been tracking (e.g., continuously or intermittently) the target for a predetermined period of time)].

In some embodiments, the functional vision test is a test (e.g., an outcome measure, e.g., a functional endpoint) for one or more eye conditions selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

In some embodiments, the tests results are an outcome measure for an eye condition (e.g., affecting one or both eyes of the subject). In some embodiments, the eye condition is selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

In some embodiments, the test results indicate presence of, severity of, and/or progression of an eye condition of the subject [e.g., diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD))].

In some embodiments, the test results comprise a metric that corresponds to functional vision of the subject (e.g., an area under curve (AUC) metric) (e.g., having at least a 90% sensitivity at 100% specificity, at least 91% sensitivity at 100% specificity, at least 92% sensitivity at 100% specificity, or at least 92.5% sensitivity at 100% specificity) (e.g., having at least a 90% sensitivity, at least 91% sensitivity, at least 92% sensitivity, or at least 92.5% sensitivity). In some embodiments, the metric is a sparse AUC metric (e.g., wherein one or more radial sweeps have not been performed and/or not considered). In some embodiments, the test results comprise a function of contrast sensitivity (e.g., inverse of root-mean-square (RMS) contrast ratio) and spatial frequency (in cycles per degree, CPD) (e.g., stored as or presented in a plot).

In some embodiments, each of the one or more targets is a visibility patch (e.g., a contrast-based visibility patch) graphically rendered within the virtual and/or augmented scene.

In some embodiments, the method is performed without use of artificial intelligence.

In some embodiments, the method includes simulating, by the processor, a scotoma during the functional vision test (e.g., using a method disclosed herein).

In one aspect, the invention is directed to a packaged pharmaceutical composition or kit comprising a pharmaceutically acceptable vessel, a therapeutic agent secured or otherwise sealed within the vessel, and a label, wherein the therapeutic agent is for an eye condition [e.g., geographic atrophy and/or an age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD))], and the label comprises a description and/or code that identifies that the therapeutic agent is for treatment of the eye condition, (i) the eye condition having been diagnosed and/or monitored by a functional vision test performed according to the method of any one of claims 1-26 and/or (ii) efficacy of the therapeutic agent having been established and/or confirmed in a population of subjects (e.g., patients) by a functional vision test performed according to the method of any one of claims 1-26.

In one aspect, the invention is directed to a method of treating a subject that has been diagnosed with an eye condition, has been or is monitored for an eye conditions, and/or has been determined to exhibit a worsening severity of an eye condition (e.g., affecting one or both eyes of the subject) using a functional vision test according to a method disclosed herein, the method comprising administering a therapeutically effective amount of a therapeutic agent (e.g., pharmaceutical compound) to the subject.

In some embodiments, the eye condition is selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

In some embodiments, the therapeutic agent is an antibody (e.g., a monoclonal antibody) (e.g., an anti-factor D antibody or a vascular endothelial growth factor (VEGF) inhibitor). In some embodiments, the therapeutic agent is a vascular endothelial growth factor (VEGF) inhibitor. In some embodiments, the therapeutic agent is ranibizumab, faricimab, brolucizumab, aflibercept, or pegaptanib. In some embodiments, the therapeutic agent comprises a vitamin supplement and/or mineral supplement (e.g., comprising vitamin C, zinc, vitamin E, copper, or beta-carotene). In some embodiments, the therapeutic agent comprises a complement inhibitor (e.g., a C3 inhibitor or a C5 inhibitor). In some embodiments, the complement inhibitor comprises a peptide, protein, antibody, or aptamer that binds to C3 and/or a biologically active fragment of C3 (e.g., C3b or C3a). In some embodiments, the therapeutic agent is (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy.

In one aspect, the invention is directed to a method of determining therapeutic effectiveness (e.g., benefit) of a therapeutic agent, the method comprising (i) administering a functional vision test according to a method disclosed herein to a subject and (ii) determining therapeutic effectiveness (e.g., benefit) of a therapeutic agent to the subject based on test results from the functional vision test. In some embodiments, determining the therapeutic effectiveness comprises quantifying quality adjusted life years (QALY). In some embodiments, the method includes determining cost effectiveness of the therapeutic agent based, at least in part, on determining the therapeutic effectiveness based on test results from the functional vision test (e.g., based on quantifying QALY). In some embodiments, the therapeutic agent is (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy.

In one aspect, the invention is directed to use of (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy, for treatment of a subject diagnosed with and/or monitored for an eye condition using a method disclosed herein.

In one aspect, the invention is directed to a method of determining therapeutic effectiveness (e.g., benefit) of a therapeutic intervention, the method comprising (i) administering a functional vision test according to a method disclosed herein and (ii) determining therapeutic effectiveness (e.g., benefit) of a therapeutic intervention (e.g., laser coagulation therapy) to a subject based on test results from the functional vision test.

In one aspect, the invention is directed to a method of treating a subject that has been diagnosed with an eye condition, has been or is monitored for an eye conditions, and/or has been determined to exhibit a worsening severity of an eye condition (e.g., affecting one or both eyes of the subject) using a functional vision test according to a method disclosed herein, the method of treating comprising administering a therapeutically effective therapeutic intervention (e.g., laser coagulation therapy) to the subject.

In one aspect, the invention is directed to a method of treating a subject that has been diagnosed with an eye condition, has been or is monitored for an eye conditions, and/or has been determined to exhibit a worsening severity of an eye condition (e.g., affecting one or both eyes of the subject), the method comprising administering to the subject a therapeutically effective amount of a therapeutic agent (e.g., pharmaceutical compound), wherein the efficacy of the therapeutic agent has been established or confirmed in a population of subjects using a functional vision test according to a method disclosed herein. In some embodiments, the eye condition is selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)). In some embodiments, treatment with the therapeutic agent preserves or improves performance on the functional vision test and/or reduces rate of deterioration in performance on the functional vision test as compared to a suitable control [e.g., no treatment or sham treatment (e.g., placebo)].

In one aspect, the invention is directed to use of a therapeutic agent for treatment of an individual diagnosed with an eye condition [e.g., geographic atrophy and/or an age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (AMD)] wherein therapeutic efficacy of the therapeutic agent has been established or confirmed using a method disclosed herein in a population of subjects. In some embodiments, the therapeutic agent comprises (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy.

In one aspect, the invention is directed to a system [e.g., a virtual- and/or augmented- and/or mixed-reality device (e.g., VR headset with eye tracking capability)], comprising a processor and a memory having instructions stored thereon, the instructions executable by the processor to perform a functional vision test by a method disclosed herein.

In one aspect, the invention is directed to a method for conducting a vision test on a subject using a virtual- and/or augmented- and/or mixed-reality device (e.g., a VR headset with eye tracking capability), the method comprising rendering and displaying to the subject, by a processor, an object within a virtual and/or augmented scene in a visual field of the subject via the device (e.g., on a head-mounted display of the VR headset), wherein a position of the object remains fixed in a virtual space (e.g., fixed within the virtual and/or augmented scene) when a head of the subject changes orientation [e.g., such that the subject can orient the object in a preferred location relative to a point of gaze of the subject (e.g., a location corresponding to alignment of best vision with an area of interest in the object)]. In some embodiments, the subject translating the device does not move the object relative to the subject in the virtual space (e.g., the subject cannot get closer or further from the object in the virtual space). In some embodiments, the object (e.g., as rendered on a head-mounted display of the VR headset) is curved (i.e., not flat) (e.g., a curved test chart) (e.g., to account for diminished peripheral vision due to quality of one or more lenses of the device). In some embodiments, the object is a virtual test chart (e.g., eye chart) (e.g., comprising one or more targets, e.g., moving target(s), e.g., for a functional vision test). In some embodiments, the VR headset is a low-cost, off-the-shelf headset.

In one aspect, the invention is directed to a system for identifying a preferred retinal locus (PRL) relative to retina anatomy (e.g., a locus in an anatomy reference system) for one or both eyes of a subject (e.g., a patient with a macular disease such as macular degeneration, e.g., a patient with a central scotoma) (e.g., wherein the preferred retinal locus is a position on the retina other than the fovea or macula), the system comprising: a processor of a computing device; and a memory having instructions stored thereon, wherein the instructions, when executed by the processor, cause the processor to: receive a first data stream (e.g., from a VR headset) corresponding to a gaze direction of an eye of the subject (e.g., said gaze direction defining a visual axis) in a headset reference system (e.g., independent of actual eye anatomy) over time; receive a second data stream (e.g., from the VR headset) corresponding to a gaze origin at a nodal point of the eye of the subject (e.g., said nodal point corresponding to a center of corneal curvature of the eye) in a headset reference system over time, wherein the nodal point moves around a center of eye rotation as the eye rotates to change gaze direction; identify a geometric volume (e.g., a sphere, e.g., a best fit sphere) having a reference point (e.g., a center) corresponding to the center of eye rotation and having a surface approximated by the nodal points, using data from the second data stream; identify an anatomical reference (e.g., an optical axis of the eye) from the identified geometric volume (e.g., the sphere, e.g., the best fit sphere) (e.g., identify the optical axis of the eye as a line connecting center of the sphere and gaze origin); and identify the preferred retinal locus (PRL) relative to retina anatomy (e.g., a locus in the anatomy reference system) using the identified anatomical reference (e.g., the optical axis of the eye) and data from the first data stream (e.g., use data from the first data stream to calculate a horizontal angle and/or vertical angle—φ, θ—between the optical axis and visual axis of the eye, and identify the PRL using the identified optical axis of the eye and the calculated horizontal angle φ and/or vertical angle θ) [e.g., and monitor the PRL (e.g., the angles φ, θ), in multiple sessions with the subject performed over time (e.g., months or years) to detect PRL changes, e.g., as disease progresses].

In certain embodiments, the system comprises a virtual- and/or augmented- and/or mixed-reality headset for producing the first data stream and the second data stream. In certain embodiments, the system comprises an eye-tracking camera (e.g., wherein the headset comprises the eye-tracking camera). In certain embodiments, the system comprises an illumination source (e.g., an infrared illumination source) for illuminating (the) one or both eyes of the subject (e.g., wherein the headset comprises the illumination source).

In certain embodiments, the virtual- and/or augmented- and/or mixed-reality headset comprises one or more members selected from the group consisting of: a head-mounted display, one or more lenses, one or more headset processors for producing the first data stream and/or the second data stream [e.g., wherein the processor of the computing device that executes the instructions is any one or more of (i) to (iv) as follows: (i) a portion or all of the one or more headset processors, (ii) distinct from the one or more headset processors, (iii) at least partially co-located with the one or more headset processors, (iv) spatially separated (remote) from the one or more headset processors, and (v) in electrical and/or data communication with the one or more headset processors], one or more mechanisms (e.g., dial, toggle, knob, or switch) for physically adjusting the position of the display in the headset, one or more mechanisms (e.g., mechanical mechanisms, e.g., knobs, straps, dials, or the like) for adjusting (e.g., manually adjusting) the horizontal and/or vertical position of the headset relative to the head of the subject, a circuit board, and a head support (e.g., strap(s), mount(s), brace(s), and/or other physical structure(s) to stabilize the headset on the head of the subject).

In certain embodiments, the instructions, when executed by the processor, cause the processor to display visual stimuli (e.g., one or more static and/or changing/moving graphical targets) to the subject to elicit and record patterns of eye movements relative to the stimuli using the identified PRL, where said recorded patterns of eye movements are correlated to a disease or condition (e.g., a retinal dysfunction). In certain embodiments, the instructions, when executed by the processor, cause the processor to identify said subject may have said disease or condition based at least in part on said recorded patterns of eye movements (e.g., and wherein the instructions, when executed by the processor, cause the processor to present a graphical display (e.g., an alphanumeric) indicating the subject may have said disease or condition).

In another aspect, the invention is directed to a system for prompting adjustment (e.g., physical adjustment, e.g., manual adjustment by the wearer) of a virtual- and/or augmented- and/or mixed-reality headset position relative to a wearer's head (e.g., to improve/optimize alignment of the center of the headset lens(es) with the center of the (respective) eye(s) of the wearer), the system comprising: a virtual- and/or augmented- and/or mixed-reality headset with one or more mechanisms (e.g., mechanical mechanisms, e.g., knobs, straps, dials, or the like) for adjusting (e.g., manually adjusting) the vertical position of the headset relative to the head of the wearer; a processor of a computing device; and a memory having instructions stored thereon, wherein the instructions, when executed by the processor, cause the processor to: receive a first data stream corresponding to wearer eye position relative to the headset (e.g., the first data stream corresponding to gaze origin at a nodal point of (each of) one or both eyes of the wearer, each said nodal point corresponding to a center of corneal curvature of the (respective) eye) (e.g., the position of the center of one or both eyes of the wearer relative to the center of one or both respective headset lenses) over time, wherein a position of a virtual camera is linked to the wearer eye position relative to the headset; and render and display a virtual iron sight in a visual field of the headset wearer in real time (or near real time) corresponding to real-time (or near real time) position of the virtual camera as the first data stream is received, said iron sight comprising two concentric rings having fixed position relative to each other and a third ring having a color and/or tint that contrasts with the two concentric rings, said third ring having a visually detectable offset relative to the two concentric rings when a center of one or both respective headset lens(es) is/are misaligned with the gaze origin(s) (center(s) of corneal curvature) of the respective eye(s) of the wearer, wherein an offset of the third ring relative to the two concentric rings as it appears to the wearer in the visual field of the headset prompts physical adjustment (e.g., by the wearer) of the vertical position of the headset via the one or more (e.g., mechanical) mechanisms until said adjustment causes the center of one or both respective headset lens(es) to be aligned with the gaze origin(s) of the respective eye(s) of the wearer, and the resulting position of the virtual camera causes the third ring to be displayed entirely between the two concentric rings. In certain embodiments, the virtual- and/or augmented- and/or mixed-reality headset produces the first data stream. In certain embodiments, the system comprises an eye-tracking camera (e.g., wherein the headset comprises the eye-tracking camera). In certain embodiments, the system comprises an illumination source (e.g., an infrared illumination source) for illuminating (the) one or both eyes of the wearer (e.g., wherein the headset comprises the illumination source).

In certain embodiments, the virtual- and/or augmented- and/or mixed-reality headset comprises one or more members selected from the group consisting of: a head-mounted display, one or more lenses, one or more mechanisms (e.g., dial, toggle, knob, or switch) for physically adjusting the position of the display in the headset, a circuit board, and a head support (e.g., strap(s), mount(s), brace(s), and/or other physical structure(s) to stabilize the headset on the head of the subject).

In certain embodiments, the instructions, when executed by the processor, cause the processor to—following said adjustment that causes the center of one or both respective headset lens(es) to be aligned with the gaze origin(s) of the respective eye(s) of the wearer—display visual stimuli (e.g., one or more static and/or changing/moving graphical targets) to the subject to elicit and record patterns of eye movements relative to the stimuli, where said recorded patterns of eye movements are correlated to a disease or condition (e.g., a retinal dysfunction). In certain embodiments, the instructions, when executed by the processor, cause the processor to identify said subject may have said disease or condition based at least in part on said recorded patterns of eye movements (e.g., and wherein the instructions, when executed by the processor, cause the processor to present a graphical display (e.g., an alphanumeric) indicating the subject may have said disease or condition).

In another aspect, the invention is directed to a system for rendering a graphical (e.g., 2D) scotoma mask that simulates the effect of a scotoma on a visual field, said scotoma suffered by a particular patient, the system comprising: a processor of a computing device; and a memory having instructions stored thereon, wherein the instructions, when executed by the processor, cause the processor to: receive data corresponding to a shape of a simulated scotoma (e.g., a selected default scotoma shape such as a disc, or a personalized scotoma shape derived from microperimetry measurement of a subject, e.g., a differential light sensitivity (DLS) map); receive input (e.g., from the particular patient suffering from a real scotoma) to identify one or more visual effects parameters corresponding to values of visual effects caused by the scotoma (e.g., said one or more parameters comprising one or more members selected from the group consisting of opacity, color saturation, blur, and distortion (e.g., pincushion, barrel, or whirl)); define the graphical scotoma mask according to the shape of the simulated scotoma and the one or more visual effects parameters; receive a first data stream (e.g., from a VR headset) corresponding to a point of gaze of the wearer of the VR headset; and render and display in real time (or near real time) to the wearer of the VR headset a virtual and/or augmented scene in the visual field of the wearer, at least a portion of said virtual and/or augmented scene modified according to the graphical scotoma mask, said virtual and/or augmented scene following a point of gaze of the wearer as said point of gaze changes in real time and as said virtual and/or augmented scene is affected by the simulated scotoma (e.g., wherein the wearer may be the patient or wherein the wearer may be an individual different from the patient).

In certain embodiments, the instructions cause the processor to identify the one or more visual effects parameters from patient input by: for a first period of time, (i) blocking vision of a healthy eye of the patient and (ii) displaying a virtual scene to the scotoma-affected eye of the patient or allowing viewing of a real field of view by the scotoma-affected eye of the patient, said patient having a one-sided scotoma; for a second period of time, rendering and displaying (e.g., via the VR headset) the virtual scene and/or an augmented scene to only a (single) healthy eye of the patient a simulated scotoma, said virtual and/or augmented scene modified according to a graphical scotoma mask corresponding to a given shape and one or more adjustable visual effects parameters; and updating the virtual and/or augmented scene according to feedback from the patient and rendering and displaying the updated virtual and/or augmented scene to the healthy eye of the patient in real time (or near real time), such that the patient may compare the patient's field of view in each eye and adjust the one or more visual effects parameters (and/or the scotoma shape) to match the field of view as seen by the eye with the real scotoma with the field of view as seen by the eye with the simulated scotoma.

In certain embodiments, the system comprises a virtual- and/or augmented- and/or mixed-reality headset which produces the first data stream.

In certain embodiments, the system comprises an eye-tracking camera (e.g., wherein the headset comprises the eye-tracking camera).

In certain embodiments, the system comprises an illumination source (e.g., an infrared illumination source) for illuminating (the) one or both eyes of the patient (e.g., wherein the headset comprises the illumination source).

In certain embodiments, the system comprises the virtual- and/or augmented- and/or mixed-reality headset comprises one or more members selected from the group consisting of: a head-mounted display, one or more lenses, one or more mechanisms (e.g., dial, toggle, knob, or switch) for physically adjusting the position of the display in the headset, one or more mechanisms (e.g., mechanical mechanisms, e.g., knobs, straps, dials, or the like) for adjusting (e.g., manually adjusting) the horizontal and/or vertical position of the headset relative to the head of the subject, a circuit board, and a head support (e.g., strap(s), mount(s), brace(s), and/or other physical structure(s) to stabilize the headset on the head of the subject).

In certain embodiments, the instructions, when executed by the processor, cause the processor to display visual stimuli (e.g., one or more static and/or changing/moving graphical targets) to the subject to elicit and record patterns of eye movements relative to the stimuli, where said recorded patterns of eye movements are correlated to a disease or condition (e.g., a retinal dysfunction). In certain embodiments, the instructions, when executed by the processor, cause the processor to identify said wearer may have (or may have a risk of) said disease or condition based at least in part on said recorded patterns of eye movements (e.g., and wherein the instructions, when executed by the processor, cause the processor to present a graphical display (e.g., an alphanumeric) indicating the wearer may have (or may have a risk of) said disease or condition).

In another aspect, the invention is directed to a method for identifying a preferred retinal locus (PRL) relative to retina anatomy (e.g., a locus in an anatomy reference system) for one or both eyes of a subject (e.g., a patient with a macular disease such as macular degeneration, e.g., a patient with a central scotoma) (e.g., wherein the preferred retinal locus is a position on the retina other than the fovea or macula), the method comprising: receiving, by a processor of a computing device, a first data stream (e.g., from a VR headset) corresponding to a gaze direction of an eye of the subject (e.g., said gaze direction defining a visual axis) in a headset reference system (e.g., independent of actual eye anatomy) over time; receiving, by the processor, a second data stream (e.g., from the VR headset) corresponding to a gaze origin at a nodal point of the eye of the subject (e.g., said nodal point corresponding to a center of corneal curvature of the eye) in a headset reference system over time, wherein the nodal point moves around a center of eye rotation as the eye rotates to change gaze direction; identifying, by the processor, a geometric volume (e.g., a sphere, e.g., a best fit sphere) having a reference point (e.g., a center) corresponding to the center of eye rotation and having a surface approximated by the nodal points, using data from the second data stream; identifying, by the processor, an anatomical reference (e.g., an optical axis of the eye) from the identified geometric volume (e.g., the sphere, e.g., the best fit sphere) (e.g., identify the optical axis of the eye as a line connecting center of the sphere and gaze origin); and identifying the preferred retinal locus (PRL) relative to retina anatomy (e.g., a locus in the anatomy reference system) using the identified anatomical reference (e.g., the optical axis of the eye) and data from the first data stream (e.g., use data from the first data stream to calculate a horizontal angle and/or vertical angle—φ, θ—between the optical axis and visual axis of the eye, and identify the PRL using the identified optical axis of the eye and the calculated horizontal angle φ and/or vertical angle θ) [e.g., and monitor the PRL (e.g., the angles (φ, θ), in multiple sessions with the subject performed over time (e.g., months or years) to detect PRL changes, e.g., as disease progresses].

In certain embodiments, the method comprises displaying visual stimuli (e.g., one or more static and/or changing/moving graphical targets) to the subject to elicit and record (e.g., by the processor) patterns of eye movements relative to the stimuli using the identified PRL, where said recorded patterns of eye movements are correlated to a disease or condition (e.g., a retinal dysfunction). In certain embodiments, the method comprises identifying, by the processor, said subject as having (e.g., or, alternatively, identifying said subject as having a risk of) said disease or condition based at least in part on said recorded patterns of eye movements (e.g., and presenting, by the processor, a graphical display (e.g., an alphanumeric) indicating the subject has (e.g., or, alternatively, may have) said disease or condition).

In another aspect, the invention is directed to a method for prompting adjustment (e.g., physical adjustment, e.g., manual adjustment by the wearer) of a virtual- and/or augmented- and/or mixed-reality headset position relative to a wearer's head (e.g., to improve/optimize alignment of the center of the headset lens(es) with the center of the (respective) eye(s) of the wearer), the method comprising: receiving, by a processor of a computing device, a first data stream corresponding to wearer eye position relative to the headset (e.g., the first data stream corresponding to gaze origin at a nodal point of (each of) one or both eyes of the wearer, each said nodal point corresponding to a center of corneal curvature of the (respective) eye) (e.g., the position of the center of one or both eyes of the wearer relative to the center of one or both respective headset lenses) over time, wherein a position of a virtual camera is linked to the wearer eye position relative to the headset, wherein the headset comprises one or more mechanisms (e.g., mechanical mechanisms, e.g., knobs, straps, dials, or the like) for adjusting (e.g., manually adjusting) the vertical position of the headset relative to the head of the wearer; and rendering and displaying, by the processor, a virtual iron sight in a visual field of the headset wearer in real time (or near real time) corresponding to real-time (or near real time) position of the virtual camera as the first data stream is received, said iron sight comprising two concentric rings having fixed position relative to each other and a third ring having a color and/or tint that contrasts with the two concentric rings, said third ring having a visually detectable offset relative to the two concentric rings when a center of one or both respective headset lens(es) is/are misaligned with the gaze origin(s) (center(s) of corneal curvature) of the respective eye(s) of the wearer, wherein an offset of the third ring relative to the two concentric rings as it appears to the wearer in the visual field of the headset prompts physical adjustment (e.g., by the wearer) of the vertical position of the headset via the one or more (e.g., mechanical) mechanisms until said adjustment causes the center of one or both respective headset lens(es) to be aligned with the gaze origin(s) of the respective eye(s) of the wearer, and the resulting position of the virtual camera causes the third ring to be displayed entirely between the two concentric rings.

In certain embodiments, the method comprises checking virtual iron sign alignment during a visual function test.

In certain embodiments, the method comprises, following said adjustment that causes the center of one or both respective headset lens(es) to be aligned with the gaze origin(s) of the respective eye(s) of the wearer, displaying (e.g., by the processor) visual stimuli (e.g., one or more static and/or changing/moving graphical targets) to the wearer to elicit and record patterns of eye movements relative to the stimuli, where said recorded patterns of eye movements are correlated to a disease or condition (e.g., a retinal dysfunction). In certain embodiments, the method comprises identifying, by the processor, that said wearer has (or may have) said disease or condition based at least in part on said recorded patterns of eye movements (e.g., and presenting a graphical display (e.g., an alphanumeric) indicating the wearer has (or may have) said disease or condition).

In another aspect, the invention is directed to a method for rendering a graphical (e.g., 2D) scotoma mask that simulates the effect of a scotoma on a visual field, said scotoma suffered by a particular patient, the method comprising: receiving, by the processor of a computing device, data corresponding to a shape of a simulated scotoma (e.g., a selected default scotoma shape such as a disc, or a personalized scotoma shape derived from microperimetry measurement of a subject, e.g., a differential light sensitivity (DLS) map); receiving, by the processor, input (e.g., from the particular patient suffering from a real scotoma) to identify one or more visual effects parameters corresponding to values of visual effects caused by the scotoma (e.g., said one or more parameters comprising one or more members selected from the group consisting of opacity, color saturation, blur, and distortion (e.g., pincushion, barrel, or whirl)); defining, by the processor, the graphical scotoma mask according to the shape of the simulated scotoma and the one or more visual effects parameters; receiving, by the processor, a first data stream (e.g., from a VR headset) corresponding to a point of gaze of the wearer of the VR headset; and rendering and displaying, by the processor, in real time (or near real time) to the wearer of the VR headset a virtual and/or augmented scene in the visual field of the wearer, at least a portion of said virtual and/or augmented scene modified according to the graphical scotoma mask, said virtual and/or augmented scene following a point of gaze of the wearer as said point of gaze changes in real time and as said virtual and/or augmented scene is affected by the simulated scotoma (e.g., wherein the wearer may be an individual different from the patient).

In certain embodiments, the method comprises identifying, by the processor, the one or more visual effects parameters from patient input by: for a first period of time, (i) blocking vision of a healthy eye of the patient and (ii) displaying a virtual scene to the scotoma-affected eye of the patient or allowing viewing of a real field of view by the scotoma-affected eye of the patient, said patient having a one-sided scotoma; for a second period of time, rendering and displaying (e.g., via the VR headset) the virtual scene and/or an augmented scene to only a (single) healthy eye of the patient a simulated scotoma, said virtual and/or augmented scene modified according to a graphical scotoma mask corresponding to a given shape and one or more adjustable visual effects parameters; and updating the virtual and/or augmented scene according to feedback from the patient and rendering and displaying the updated virtual and/or augmented scene to the healthy eye of the patient in real time (or near real time), such that the patient may compare the patient's field of view in each eye and adjust the one or more visual effects parameters (and/or the scotoma shape) to match the field of view as seen by the eye with the real scotoma with the field of view as seen by the eye with the simulated scotoma.

In certain embodiments, the method comprises displaying (e.g., by the processor) visual stimuli (e.g., one or more static and/or changing/moving graphical targets) to the wearer to elicit and record patterns of eye movements relative to the stimuli, where said recorded patterns of eye movements are correlated to a disease or condition (e.g., a retinal dysfunction). In certain embodiments, the method comprises identifying, by the processor, said wearer may have (or may have a risk of) said disease or condition based at least in part on said recorded patterns of eye movements (e.g., and presenting a graphical display (e.g., an alphanumeric) indicating the wearer may have (or may have a risk of) said disease or condition).

In another aspect, the invention is directed to a system for conducting a functional vision test (e.g., a contrast sensitivity and/or spatial frequency test, e.g., a radial sweep test) on a subject using a virtual- and/or augmented- and/or mixed-reality device (e.g., VR headset with eye tracking capability), the system comprising: a processor of a computing device; and a memory having instructions stored thereon, wherein the instructions, when executed by the processor, cause the processor to: render and display (e.g., via the VR headset) to the subject, over a course of the functional vision test, a target (e.g., a moving target, e.g., a target that changes in spatial frequency and contrast at discrete intervals, e.g., along a plurality of sweep trajectories, e.g., where the sweeps all begin with the target at a common origin that then radiate outward along vectors in contrast sensitivity function (CSF) space until they reach the limit of function, at which point target invisibility prevents further tracking by the subject and a threshold is recorded); automatically perform, in real time, during the course of the functional vision test, a visibility determination using a machine learning algorithm to automatically identify if the subject is accurately tracking the target in time and space; and compute and/or display test results using the visibility determination.

In certain embodiments, the test results comprise a plot of contrast sensitivity (e.g., inverse of root-mean-square (RMS) contrast ratio) and spatial frequency (in cycles per degree, CPD).

In certain embodiments, the machine learning algorithm (e.g., said machine learning algorithm comprising one or more recombinant neural networks, and/or long short-time memory, and/or one or more temporal convolutional networks) has been previously trained from user trials with ground truth established by manual assessment of video recordings of the users that determined if a subject eye position was within an individual target (e.g., culled from hundreds of minutes of observation and annotation).

In certain embodiments, the machine learning algorithm determines, during the course of the functional vision test, a probability that, for a given time window (e.g., in milliseconds, e.g., <250 milliseconds, <100 milliseconds, <50 milliseconds, <25 milliseconds, etc.) the subject is (or is not) observing the target (e.g., an individual target in a field of a plurality of targets, e.g., 3 or more targets, e.g., 5 targets), wherein appearance of the target as presented to the subject is altered (e.g., value of contrast and/or value of spatial frequency) at least once during the course of the functional vision test upon determination the subject is (likely) observing the target (e.g., wherein the appearance of the target stops being altered when the algorithm determines the subject is no longer tracking the target, wherein final values of contrast and spatial frequency are recorded as the subject's visual/functional threshold for that parameter space, e.g., at which point a new target is presented and the process is repeated, e.g., a new sweep is conducted).

In certain embodiments, the instructions, when executed by the processor, cause the processor to adjust one or more threshold parameters (e.g., during the course of the functional vision test) (e.g., wherein the one or more threshold parameters comprises one or both of (i) and (ii) as follows: (i) a tolerance for how near or far the subject's actual eye position is located from the center of the target, e.g., the circular target, and (ii) a time window for the visibility determination using the machine learning algorithm.

In another aspect, the invention is directed to a method for conducting a functional vision test (e.g., a contrast sensitivity and/or spatial frequency test, e.g., a radial sweep test) on a subject using a virtual- and/or augmented- and/or mixed-reality device (e.g., VR headset with eye tracking capability), the method comprising: rendering and displaying (e.g., via the VR headset) to the subject, by a processor of a computing device, over a course of the functional vision test, a target (e.g., a moving target, e.g., a target that changes in spatial frequency and contrast at discrete intervals, e.g., along a plurality of sweep trajectories, e.g., where the sweeps all begin with the target at a common origin that then radiate outward along vectors in contrast sensitivity function (CSF) space until they reach the limit of function, at which point target invisibility prevents further tracking by the subject and a threshold is recorded); automatically performing, by the processor, in real time, during the course of the functional vision test, a visibility determination using a machine learning algorithm to automatically identify if the subject is accurately tracking the target in time and space; and computing and/or displaying, by the processor, test results using the visibility determination.

In certain embodiments, the test results comprise a plot of contrast sensitivity (e.g., inverse of root-mean-square (RMS) contrast ratio) and spatial frequency (in cycles per degree, CPD).

In certain embodiments, the machine learning algorithm (e.g., said machine learning algorithm comprising one or more recombinant neural networks, and/or long short-time memory, and/or one or more temporal convolutional networks) has been previously trained from user trials with ground truth established by manual assessment of video recordings of the users that determined if a subject eye position was within an individual target (e.g., culled from hundreds of minutes of observation and annotation).

In certain embodiments, the machine learning algorithm determines, during the course of the functional vision test, a probability that, for a given time window (e.g., in milliseconds, e.g., <250 milliseconds, <100 milliseconds, <50 milliseconds, <25 milliseconds, etc.) the subject is (or is not) observing the target (e.g., an individual target in a field of a plurality of targets, e.g., 3 or more targets, e.g., 5 targets), wherein appearance of the target as presented to the subject is altered (e.g., value of contrast and/or value of spatial frequency) at least once during the course of the functional vision test upon determination the subject is (likely) observing the target (e.g., wherein the appearance of the target stops being altered when the algorithm determines the subject is no longer tracking the target, wherein final values of contrast and spatial frequency are recorded as the subject's visual/functional threshold for that parameter space, e.g., at which point a new target is presented and the process is repeated, e.g., a new sweep is conducted).

In certain embodiments, the method comprises adjusting, by the processor, one or more threshold parameters (e.g., during the course of the functional vision test) (e.g., wherein the one or more threshold parameters comprises one or both of (i) and (ii) as follows: (i) a tolerance for how near or far the subject's actual eye position is located from the center of the target, e.g., the circular target, and (ii) a time window for the visibility determination using the machine learning algorithm.

In certain embodiments, the method comprises features of any of the other methods described herein.

Any two or more of the features described in this specification, including in this summary section, may be combined to form implementations of the disclosure, whether specifically expressly described as a separate combination in this specification or not.

At least part of the methods, systems, and techniques described in this specification may be controlled by executing, on one or more processing devices, instructions that are stored on one or more non-transitory machine-readable storage media. Examples of non-transitory machine-readable storage media include read-only memory, an optical disk drive, memory disk drive, and random access memory. At least part of the methods, systems, and techniques described in this specification may be controlled using a computing system comprised of one or more processing devices and memory storing instructions that are executable by the one or more processing devices to perform various control operations.

### DEFINITIONS

In order for the present disclosure to be more readily understood, certain terms used herein are defined below. Additional definitions for the following terms and other terms may be set forth throughout the specification.
Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.
Therapeutically effective: As used herein, a therapeutically effective substance [e.g., therapeutic agent (e.g., pharmaceutical compound, for example in a formulation)] is one that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective substance is one that, when administered in an appropriate amount, reduces the incidence and/or severity of, and/or delays onset of, one or more symptoms of the disease, disorder, and/or condition. Those of ordinary skill in the art will appreciate that the term “therapeutically effective” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.
Treatment: As used herein, the term “treatment” (also “treat” or “treating”) refers to any administration of a therapeutic compound or therapeutic procedure (e.g., surgical intervention) that partially or completely alleviates, ameliorates, relives, inhibits, delays onset of, reduces severity of, and/or reduces incidence of one or more symptoms, features, and/or causes of a particular condition (e.g., disease or disorder). In some embodiments, such treatment may be of a subject who does not exhibit signs of the relevant condition (e.g., disease or disorder) and/or of a subject who exhibits only early signs of the condition (e.g., disease or disorder). Alternatively or additionally, such treatment may be of a subject who exhibits one or more established signs of the relevant d condition (e.g., disease or disorder). In some embodiments, treatment may be of a subject who has been diagnosed as suffering from the relevant condition (e.g., disease or disorder). In some embodiments, treatment may be of a subject known to have one or more susceptibility factors that are statistically correlated with increased risk of development of the relevant condition (e.g., disease or disorder).
Subject: As used herein “a subject” is a human. In some embodiments, a subject is or may be suffering from a relevant condition (e.g., disease or disorder), for example of the eye (e.g., in one or both eyes). In some embodiments, a subject is susceptible to a condition (e.g., disease or disorder), such as an eye condition. In some embodiments, a subject displays one or more symptoms or characteristics of a condition (e.g., disease or disorder). In some embodiments, a subject does not display any symptom or characteristic of a condition (e.g., disease or disorder). In some embodiments, a subject is someone with one or more features characteristic of susceptibility to or risk of a condition (e.g., disease or disorder). In some embodiments, a subject is a patient. In some embodiments, a subject is an individual to whom diagnosis and/or a therapeutic agent is, may be, and/or has been administered. A subject may be diagnosed with and/or monitored for an eye condition. An eye condition of a subject may be monitored. An eye condition may affect one or both eyes of a subject. In some embodiments, a condition is an eye condition, such as, for example, diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)). Other eye conditions that affect sight (e.g., functional vision) are known in the art.
Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, intravenous, parenteral, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIGS. 1A and 1B are schematic diagrams of a system and method for identifying a preferred retinal locus (PRL) relative to retina anatomy using a VR headset, according to an illustrative embodiment.

FIG. 2 is a block flow diagram of an exemplary method for identifying a PRL using a VR headset system, according to an illustrative embodiment.

FIGS. 3A and 3B are images of views through VR headset lenses having aberrations that are more pronounced in the periphery than in the center.

FIG. 4 is a block flow diagram of an exemplary method for prompting adjustment of the vertical position of a VR headset such that the position of the center of one or both eyes of the wearer relative to the center of one or both respective VR headset lenses is/are aligned, according to an illustrative embodiment.

FIG. 5 is a schematic diagram illustrating virtual iron sights used in a method for prompting adjustment of a VR headset to vertically align eye and lens, according to an illustrative embodiment.

FIG. 6 is a fundus image of a central scotoma in a patient (left) and overlaid differential light sensitivity (DLS) values, for use in a system for rendering a graphical scotoma mask, according to an illustrative embodiment.

FIG. 7 is an image of a scotoma mask overlaid on newsprint, to simulate how a patient suffering from a scotoma views the newsprint, according to an illustrative embodiment.

FIG. 8A is a block flow diagram of a method for creating a scotoma mask with the help of input from a patient wearing a VR headset with eye tracking capability, according to an illustrative embodiment. In this block diagram, a “one-sided” scotoma refers to a scotoma in one eye, with unimpaired vision in the fellow eye. For this flow, the scotoma would be present in the right eye, and the left is “healthy” and is used as the comparator.

FIG. 8B is a block flow diagram of a method for rendering the scotoma mask created via the method of FIG. 8A, according to an illustrative embodiment.

FIGS. 9A-9C are schematic diagrams illustrating components of a VR headset with eye tracking capability, for use in the systems and methods described herein, according to an illustrative embodiment.

FIG. 10 is a block flow diagram of a method for identifying a preferred retinal locus (PRL) relative to retina anatomy, according to an illustrative embodiment.

FIG. 11 is a block flow diagram of a method for prompting adjustment of a VR headset position relative to a wearer's head to improve/optimize alignment of the center of the VR headset lens(es) with the center of the respective eye(s) of the wearer, according to an illustrative embodiment.

FIG. 12 is a block flow diagram of a method for rendering a graphical scotoma mask that simulates the effect of a scotoma on a visual field, according to an illustrative embodiment.

FIG. 13 is a block diagram of an exemplary cloud computing environment, in accordance with illustrative embodiments.

FIG. 14 is a schematic depicting an example of a computing device 500 and a mobile computing device 550 that can be used to perform the methods described herein, and/or can be used in the systems described herein, in accordance with illustrative embodiments.

FIGS. 15A-15B are schematics of a neural network implementation for an AI-supported radial sweep functional vision test, including a visibility determination to automatically determine whether the subject is looking at a target, according to an illustrative embodiment.

FIG. 16 are graphs indicating patch visibility probability (top panel), patch prediction (middle panel), and global visibility after applying the prediction threshold (bottom panel) in the AI-supported radial sweep functional vision test, according to an illustrative embodiment.

FIG. 17 is a graph depicting results using long short-time memory (LSTM) and temporal convolutional network (TCN) as architecture (e.g., a 1 dimensional convolutional network architecture) for the machine learning algorithm in the AI-supported radial sweep functional vision test, according to an illustrative embodiment.

FIG. 18A is a schematic illustration of an eye having a point of gaze directed at one target of multiple targets that are rendered and displayed on a virtual and/or augmented scene in a visual field of a subject, according to an illustrative embodiment.

FIG. 18B is a schematic illustration of multiple targets rendered and displayed on a virtual and/or augmented scene that may be viewed by a subject (e.g., wearing a VR headset with eye tracking), according to an illustrative embodiment.

FIG. 19A illustrates test results of a radial sweep test where the shaded regions represent regions not considered by a metric, according to an illustrative embodiment.

FIG. 19B illustrates an area under contrast sensitivity space of a radial sweep test (written as “AUC”) metric for multiple eyes having or simulating varying severity of scotoma (none, mild, or severe) showing highly reproducible and sensitive results, according to an illustrative embodiment.

FIG. 19C illustrates additional calculated AUC metric values and corresponding receiver operating characteristic (ROC) curves for a baseline and sparse metric, according to an illustrative embodiment.

### DETAILED DESCRIPTION OF CERTAIN EMBODIMENTS

Eye tracking or gaze estimation systems track eye movements to continuously estimate where on a surface or in a volume of space a subject is looking. Head-mounted eye tracking systems include dedicated camera hardware designed for capturing images of one or both eyes of the subject, and a light source (e.g., an infrared illuminator) to illuminate the eye(s). A head-mounted system may allow the subject to view a real world scene, a fully computer-generated scene (virtual reality, VR), a real-world scene with overlaid computer-generated graphical components (augmented reality, AR), and/or a mixed reality (MR) scene that merges VR and AR. The system may also offer the ability to switch between multiple of these operational modes (real-world, VR, AR, and/or MR). Moreover, the system may conduct eye tracking while an interactive VR, AR, or MR visual experience is presented to the subject such that the system identifies where in the presented virtual or augmented scene the subject is looking at any given time.

The systems and methods described herein make use of such VR headset systems (as used hereinafter, the terms “headset” or “VR headset” refer broadly to head- or face-worn systems that implement virtual reality, augmented reality, and/or mixed reality functionality).

In various embodiments, the systems and methods described herein: (i) utilize an anatomical reference to determine the preferred retinal locus (PRL) of a subject's eye, (ii) align VR headset optics with the wearer's eye, thereby reducing inaccuracy due to aberrations (e.g., chromatic, spheric) near the periphery of VR headset lenses, and/or (iii) render a graphical (e.g., 2D) scotoma mask that simulates the effect of a particular patient's scotoma on a visual field of either the patient (e.g. the patient's good eye) or another individual.

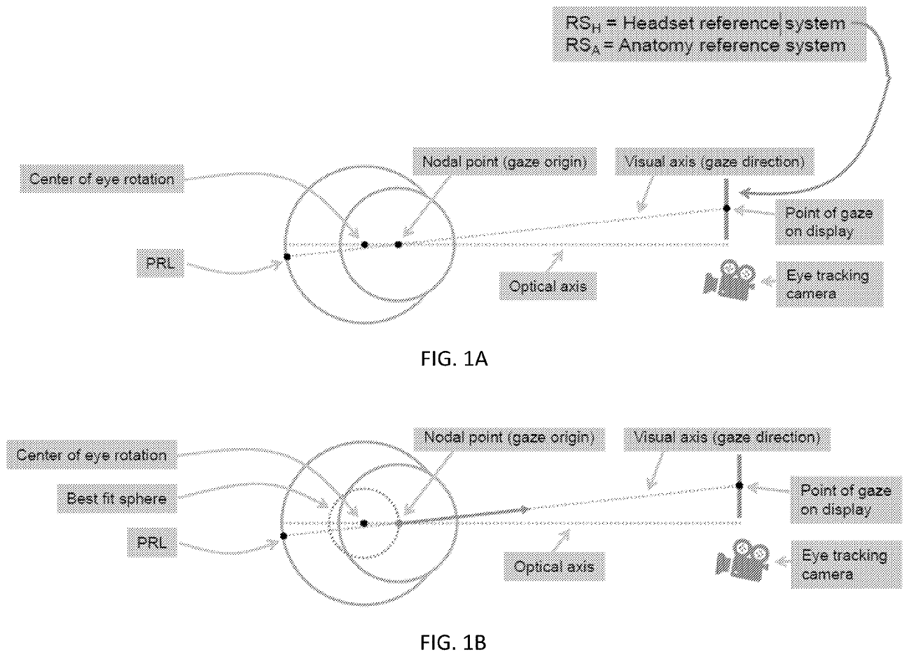

FIGS. 1A and 1B are schematic diagrams of a system and method for identifying a preferred retinal locus (PRL) relative to retina anatomy using a VR headset, according to an illustrative embodiment. Existing VR headsets measure and provide the direction of gaze in a headset reference system (RS_{H}), independent of eye anatomy (RS_{A}). The techniques described herein determine a stable reference for the anatomy of the eye in the RS_{H} headset reference system to determine the preferred retinal locus (PRL) relative to the retina anatomy. In this example, the anatomical reference would be the optical axis of the eye.

VR headsets with eye tracking capability provide gaze direction and gaze origin data streams in the headset reference system (RS_{H}). Anatomically, the gaze origin at a given point in time corresponds to a nodal point of the eye, which is the center of corneal curvature. When the eye rotates to change gaze direction, the nodal point moves around the center of eye rotation. The center of eye rotation is determined from a sufficient number of different nodal point positions. For example, these points, over time, will approximate the shape of the eye, which can be modeled as a sphere, for example, on which all nodal points lie. The optical axis of the eye is then determined from the center of rotation and nodal point.

Thus, a first stream of data in headset reference coordinates (a coordinate system independent of actual eye anatomy) is received from the VR headset corresponding to a gaze direction of an eye of the subject over time, said gaze direction defining a visual axis. A second stream of data in the headset reference coordinate system is received from the VR headset corresponding to a gaze origin at a nodal point of the eye of the subject, which changes over time. Each nodal point corresponds to the center of corneal curvature of the eye as measured at a given point in time. A geometric volume is then modeled/defined (e.g., a sphere, e.g., a best fit sphere) having a center corresponding to the center of eye rotation and having a surface approximated by the nodal points, using data from the second data stream. The optical axis of the eye is then identified from the modeled sphere, where the optical axis is a line connecting the center of the sphere and the gaze origin. Then, the preferred retinal locus (PRL) is identified relative to retina anatomy (e.g., in an eye coordinate system) using the thusly identified optical axis and data from the first stream (visual axis/gaze direction). For example, data from the first stream is used to calculate a horizontal angle and/or vertical angle—φ, θ—between the optical axis and visual axis of the eye, and the PRL is identified using the identified optical axis of the eye and the calculated horizontal angle φ and/or vertical angle φ. In certain embodiments, the method is used to monitor the PRL (e.g., the angles φ, θ) over time, for example, obtained in multiple sessions conducted at regular checkups to monitor PRL changes, for example, as the disease or condition progresses.

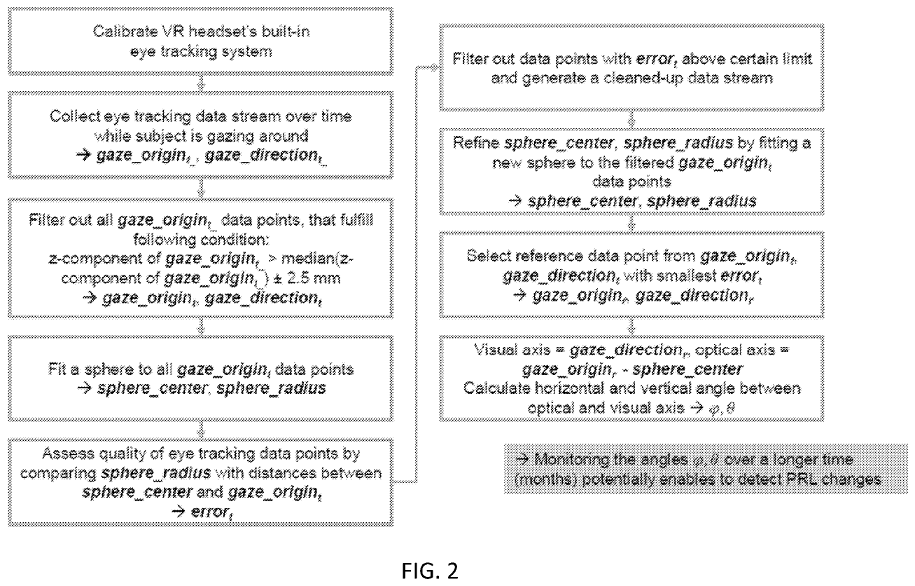

FIG. 2 is a block flow diagram of a specific exemplary method for identifying a PRL using a VR headset system, according to an illustrative embodiment. In the first step, the VR headset's built-in eye tracking system is calibrated. For example, the vertical and/or horizontal position of the lenses is adjusted using the virtual iron sight described in further detail herein below. Then, eye tracking data stream is collected over time while the subject is gazing around wearing the VR headset. The eye tracking data in this example is denoted in the FIG. 2 block diagram as gaze_originₜ and gaze_directionₜ. Next, the system filters out all gaze_originₜ data points that fulfill the condition shown, to improve data quality. Then, the system fits a sphere to the remaining gaze_originₜ data points, resulting in the determination of sphere_center and sphere_radius. The quality of eye tracking data points is assessed by comparing sphere_radius with distances between sphere_center and gaze_originₜ (difference is errorₜ). The system filters out data points with errorₜ above a given threshold, and a cleaned-up data stream is generated. The previously identified sphere_center and sphere_radius is refined by fitting a new sphere to the filtered gaze_originₜ data points. Then, a reference data point is selected from gaze_originₜ and gaze_directionₜ with smallest errorₜ. The reference data point is denoted in FIG. 2 as gaze_originᵣ (optical axis) and gaze_directionᵣ (visual axis). The system then calculates the horizontal and vertical angle between optical and visual axis, φ, θ. The system can monitor changes in these angles over time, for example, obtained in multiple sessions conducted at regular checkups. to monitor PRL changes in the patient, for example, indicating progression of disease and/or benefit gained by a prescribed therapy, surgery, and/or therapeutic.

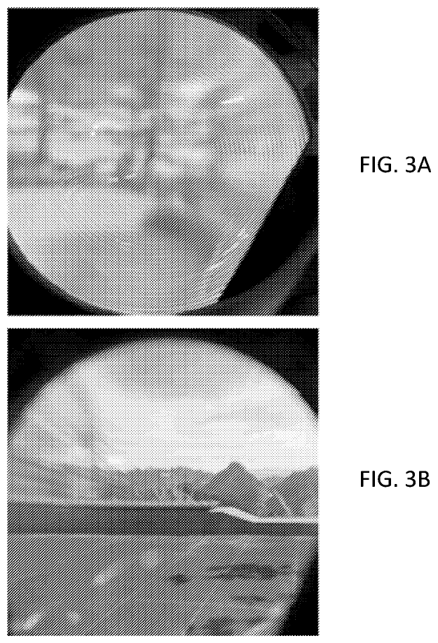

FIGS. 3A and 3B are images of views through VR headset lenses having aberrations that are more pronounced in the periphery than in the center. The optical quality of commercial VR headsets—even professional systems—is limited due to the compact lens design, for example, as seen with the use of Fresnel lenses and singlets. Singlets are generally thicker, heavier, and more curved, compared to Fresnel lenses. However, both kinds have aberrations, for example, chromatic and/or spheric aberrations, that are usually more pronounced at the periphery of the lens than in the center of the lens. This can result in inaccurate measurements when performing functional vision tests using a VR system.

Thus, certain embodiments of the systems and methods described herein implement virtual iron sights to trigger headset adjustment by the wearer (or, adjustment by an assistant of the wearer who is viewing a screen that shows the virtual image as viewed by the headset wearer), allowing interactive feedback of the wearer of the VR system to align headset optics with the eye in real time. This enables improved optical quality and more accurate measurements from functional vision tests.

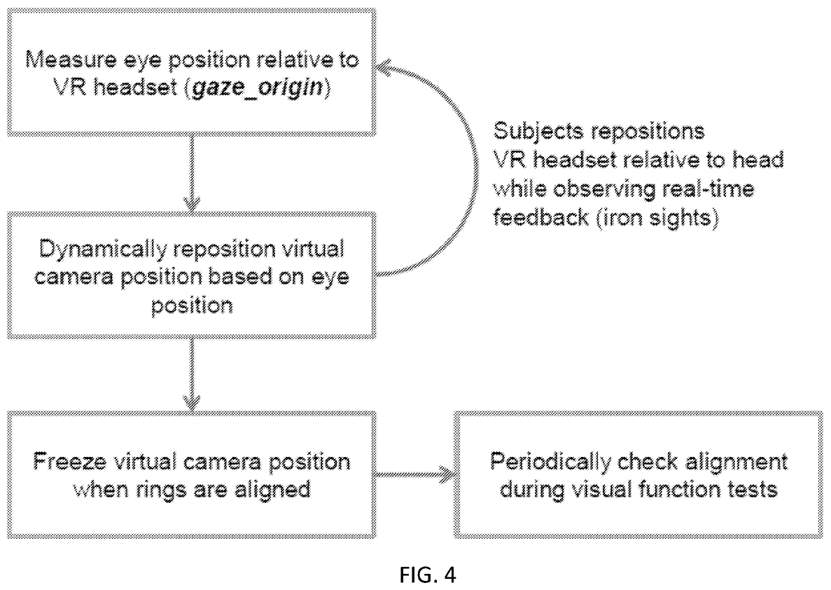

FIG. 4 is a block flow diagram of an exemplary method for prompting adjustment of the vertical position of a VR headset such that the position of the center of one or both eyes of the wearer relative to the center of one or both respective VR headset lenses is/are aligned, according to an illustrative embodiment. The system measures eye position relative to the VR headset over time, for example, the gaze_origin corresponding to a nodal point of the eye or each eye of the wearer, a nodal point corresponding to a center of corneal curvature of the respective eye. A position of a virtual camera is linked to the wearer eye position relative to the VR headset. The system then dynamically positions (and repositions) the virtual camera position based on eye position in real time. The subject repositions the VR headset while observing real-time feedback via a virtual iron sight by physically adjusting a toggle, strap, dial, switch, knob, dial, or other mechanism (e.g., manual or electronic). The gaze_origin continues to be tracked in real time and the virtual camera is dynamically repositioned as the subject repositions the VR headset. The subject continues to reposition the headset as prompted by the virtual iron sight until alignment of the virtual iron sight is achieved, indicating alignment of the centers of the eye (or eyes) of the wearer and the respective lens(es) of the VR headset.

In one example, the virtual iron sight presented in a visual field of the headset wearer in real time comprises two concentric rings having fixed position relative to each other, and a third ring having a color that contrasts with the concentric rings. The third ring has a visually detectable offset relative to the two concentric rings when a center of one or both headset lens(es) is/are misaligned with the gaze origin of the respective eye(s) of the wearer. The offset prompts physical adjustment of the headset by the wearer, thereby adjusting the vertical position of the headset until the adjustment causes the center of the lens(es) to be aligned with the gaze origin(s) of the respective eye(s) of the wearer. The resulting position of the virtual camera causes the third ring to be displayed entirely between the two concentric rings. The system can then freeze the virtual camera position when the rings are aligned. Alignment can then be periodically checked during visual function tests to insure alignment continues to be maintained.

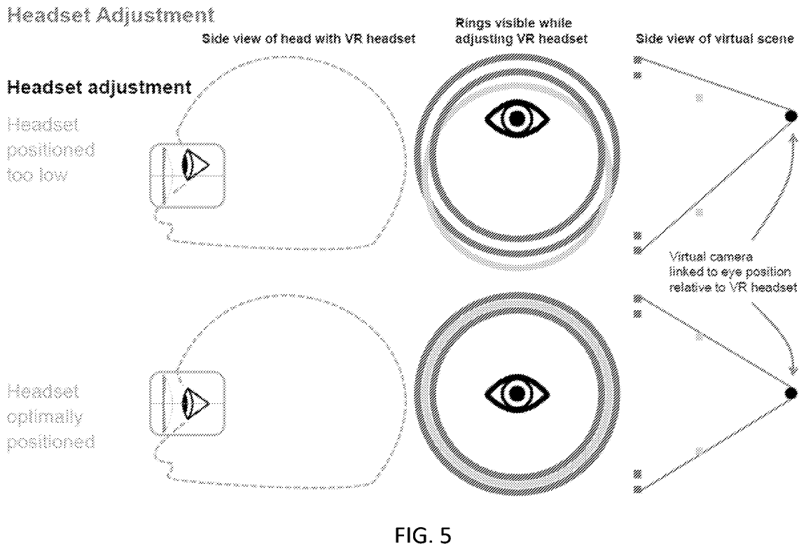

FIG. 5 is a schematic diagram illustrating virtual iron sights used in a method for prompting adjustment of a VR headset to vertically align eye and lens, according to an illustrative embodiment. In this example, the top image of a head wearing a VR headset is indicative of the headset being positioned too low on the wearer's head, causing a vertical misalignment between the center of the eye and the center of the respective lens of the VR headset. The two concentric rings with fixed space between the rings appears in blue, and the third ring appears in gold. At right, a side view of the virtual scene is depicted where the virtual camera is linked to eye position relative to the VR headset. In the top example, the headset is positioned too low, and the gold ring is below where it should be between the two blue rings for optimal vertical positioning. In the bottom example, the headset is optimally positioned. The headset has been adjusted to fit higher on the wearer's face such that the center of the lens aligns with the center of the eye. Accordingly, the gold ring appears to the wearer to fit completely between the two concentric blue rings, indicating alignment has been achieved.

Virtual iron sights other than the rings shown in FIG. 5 can be used, as long as they show a graphical offset to cue adjustment of the headset on the wearer's head/face is needed. The system may be calibrated prior to use such that the position of the virtual camera is accurately linked to the wearer eye position relative to the VR headset, and/or so that offsets presented to the wearer of the VR headset during adjustment accurately indicate relative displacement between the center of the VR headset lens(es) and the center of the wearer's eye(s). The example shown indicates vertical alignment. In other embodiments, the system displays a virtual iron sight to depict an offset to indicate to the wearer that physical adjustment of the headset is needed to achieve proper horizontal alignment of the eye(s) with the respective lens(es), for example, in addition to or instead of the vertical alignment shown in FIG. 5. In certain embodiments, there may be multiple virtual iron sights.

In certain embodiments, the systems and methods presented herein visually simulate the effect of a scotoma on the field of vision of a VR headset wearer, for example, during a functional vision test. A real scotoma is a blind spot or distortion that affects the patient's field of vision. Types of scotomas include (i) scintillating scotomas, which cause blurred vision in portions of the visual field and may have a luminous “aura” appearance, (ii) central scotomas, which are blind spots directly in the line of sight, and (iii) paracentral scotomas, which cause relative or total vision loss within 10 degrees of fixation and are not directly in the line of sight. Patient-reported scotoma features include blur, distortion, and/or absent or missing objects in the field of view. The operator of the system for scotoma simulation described herein can adjust each of these features independently to optimize the appearance of the scotoma, as informed/described by the patient.

The effect of a scotoma on the vision of an affected patient has been difficult to describe or demonstrate heretofore. The systems and methods described herein allow for a better understanding of how a scotoma affects a patient's vision by enabling simulation of the effects of the scotoma for viewing with a VR headset. This more accurate demonstration may be used, for example, to simulate the projected appearance of the field of vision due to scotoma over time if no intervention is conducted, thereby helping to inform the patient of the importance of treatment options and/or to increase patient compliance with prescribed therapy, surgery, and/or therapeutic agent(s). The ability to simulate the effect of scotoma may also allow a loved one and/or caregiver of the patient to experience how the patient views the world, thereby increasing empathy for the patient and increasing the ability to determine what activities are safe for the patient (e.g., driving). The “simulated scotoma” may also enable measurement of the sensitivity of certain visual function tests during development. For example, test results of healthy subjects performed both with and without scotoma simulation can be compared, such that results can be used to indicate the sensitivity of the functional test for identification of scotoma, for example.

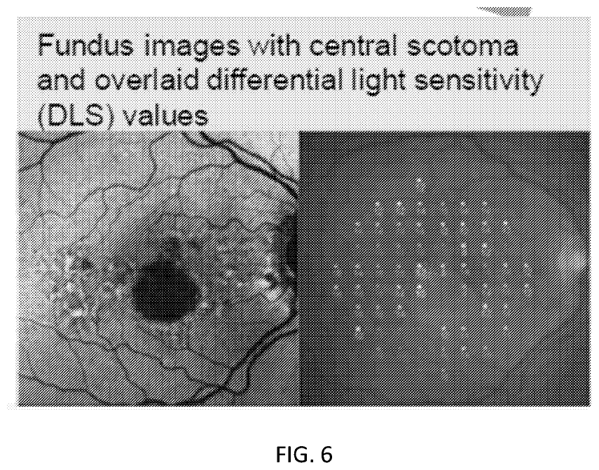

The shape of a simulated scotoma may be derived from a microperimetry measurement, for example, a differential light sensitivity (DLS) map. FIG. 6 is a fundus image of a central scotoma in a patient (left) and overlaid DLS values. Effects caused by scotoma which are often reported by affected patients include reduced opacity and color saturation, as well as increased blur and distortions (e.g., pincushion, barrel, whirl). These effects can be simulated with a VR headset, and the intensity of these affects can be individually adjusted in creating the simulated scotoma. For example, if a subject has one normal eye and one eye affected by scotoma, feedback from the subject can be solicited by presenting a virtual scene to the subject and permitting vision of the scene by one eye at a time. Feedback from the subject is then used to build the simulated scotoma (a graphical mask). The graphical mask can be rendered and displayed in real time to a wearer of the VR headset who can look around the virtual scene, which will be affected by the overlaid graphical scotoma mask. The simulated scotoma follows the point of gaze of the wearer of the VR headset in real time.

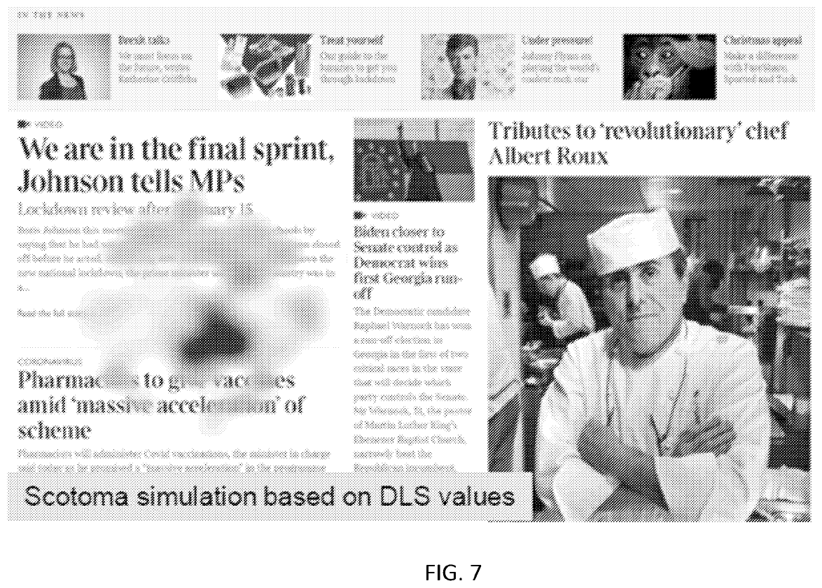

For example, FIG. 7 is an image of a scotoma mask overlaid on newsprint, to simulate how a patient suffering from a scotoma views the newsprint, according to an illustrative embodiment.

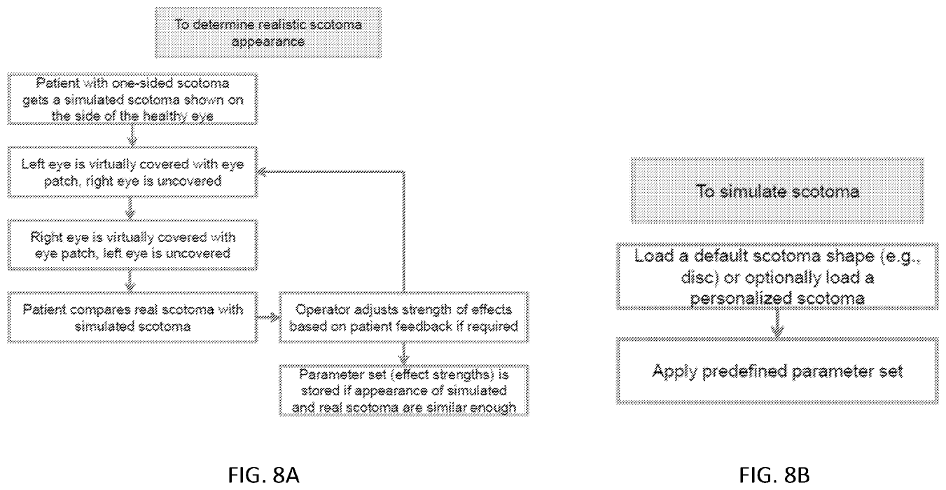

FIG. 8A is a block flow diagram of a method for creating a scotoma mask with the help of input from a patient wearing a VR headset with eye tracking capability, according to an illustrative embodiment. In this block diagram, a “one-sided” scotoma refers to a scotoma in one eye, with unimpaired vision in the fellow eye. For this flow, the scotoma would be present in the right eye, and the left is “healthy” and is used as the comparator. A simulated scotoma is shown only on the side of the healthy eye. The impaired (left) eye is virtually covered and the right eye is uncovered, able to view the virtual (or augmented/mixed) scene as affected by the virtual scotoma mask with unadjusted parameters. Then, the healthy (right) eye is virtually covered, and the impaired (left) eye is uncovered (affected by real scotoma). The patient compares the real scotoma with the simulated scotoma, and the operator adjusts the strength of effects based on patient feedback. The process repeats with each eye being covered consecutively so that the patient can compare their vision using each eye, and the strength of visual effects of the virtual scotoma continues to be adjusted until the view using the healthy eye is close to the view using the impaired eye. Then, the parameter set (effect strengths) is stored when the appearance of the simulated and real scotoma are similar enough. The scotoma mask can then be presented to a loved one, for example, to show how the patient views a virtual (or augmented/mixed) scene. A simulation of progression of the scotoma mask could also be conducted to demonstrate to the patient what the effect of the scotoma would be in a certain period of time if left untreated.

FIG. 8B is a block flow diagram of a method for rendering the scotoma mask created via the method of FIG. 8A, according to an illustrative embodiment. In this example, either a default scotoma shape or a personalized scotoma base is loaded. The personalized scotoma base may be determined, for example, from microperimetry measurement (e.g., a DLS map). A predefined parameter set is then applied. The resulting simulated scotoma can then be refined, for example, via the workflow shown in FIG. 8A.

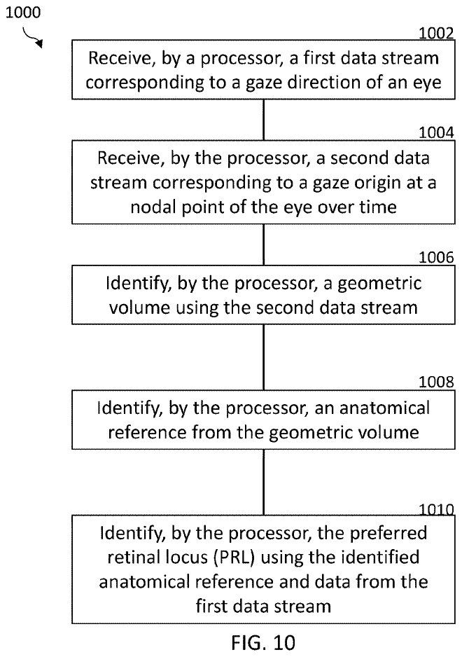

FIG. 10 illustrates an exemplary method 1000 for identifying a preferred retinal locus (PRL) relative to retina anatomy for one or both eyes of a subject (e.g., a patient). In the method 1000, data that is received in data streams over time in a headset reference system (e.g., where coordinates correspond to a virtual space for a VR headset) is oriented relative to a subject's anatomy to identify the PRL relative to retina anatomy for the subject (e.g., by converting to an anatomical reference system where coordinates correspond to physical space). In step 1002, a processor receives a first data stream corresponding to a gaze direction of an eye over time. The first data stream is oriented in a headset reference system (e.g., a virtual coordinate system independent of actual eye anatomy). In step 1004, the processor receives a second data stream corresponding to a gaze origin at a nodal point of the eye over time. The second data stream is also oriented in the headset reference system. Both the first data stream and the second data stream may be received from an apparatus or system that monitor's the subject's eye(s), for example a VR headset (embodiments of which are described in detail subsequently).

The nodal point may move around a center of eye rotation as the eye rotates to change gaze direction, for example as determined by the VR headset, such that a set of nodal temporally resolved points is determined as the subject's gaze changes direction over time. The nodal point may correspond to a center of corneal curvature of the eye.

In step 1006, the processor identifies a geometric volume using the second data stream. The geometric volume has a reference point (e.g., center) that corresponds to the center of eye rotation and a surface approximated by the nodal point(s). The geometrical volume may be, for example, a sphere, such as a best fit sphere. In step 1008, an anatomical reference (e.g., an optical axis of the eye) is identified by the processor from the identified geometric volume. In some embodiments, where the anatomical reference is an optical axis of the eye, the optical axis of the eye may be taken as a line connecting a center of the geometric volume (e.g., sphere) and a gaze origin.

In step 1010, the preferred retinal locus (PRL) is determined relative to retina anatomy using the anatomical reference and data from the first data stream. Thus, the PRL identified can be oriented relative to an anatomy reference system that may use coordinates corresponding to physical space (e.g., with a position corresponding to the eye as the origin). For example, data from the first data stream may be used to calculate a horizontal angle and/or vertical angle between the optical axis and visual axis of the eye and, thus, the PRL may be identified using the identified optical axis and the calculated horizontal angle. The PRL may be monitored (e.g., over multiple sessions performed over a longer period of time, such as weeks or months or years) to detect PRL changes, which may be indicative of disease progression.

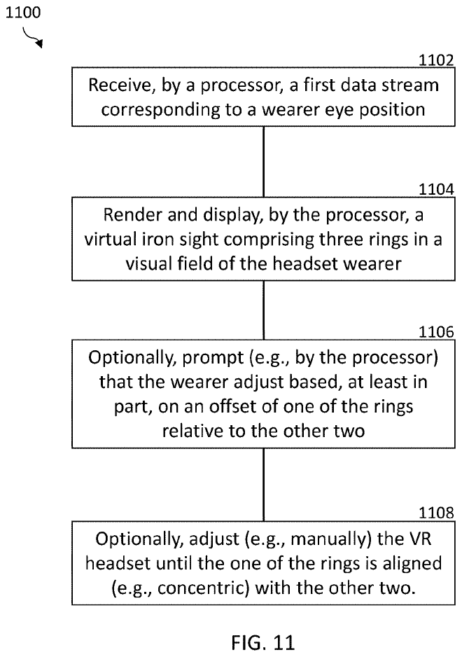

FIG. 11 illustrates an exemplary method 1100 for prompting adjustment of a VR headset position relative to a wearer's head. In step 1102, a processor receives a first data stream (over time) corresponding to a wearer eye position relative to the headset where a position of a virtual camera is linked to the wearer eye position relative to the headset. For example, the first data stream may correspond to a gaze origin at a nodal point of (each of) one or both eyes of the wearer, such as a center of corneal curvature of the (respective) eye. For example, each nodal point may correspond to the position of the center of one or both eyes of the wearer relative to the center of one or both respective headset lenses. The headset may comprise one or more adjustment mechanisms for adjusting a position (e.g., vertical and/or horizontal position) of the headset relative to the head of the wearer.

In step 1104, a virtual iron sight is rendered and displayed in a visual field of the headset wearer in real time (or near real time) corresponding to real-time (or near real time) position of the virtual camera as the first data stream is received. In certain embodiments, as in the method 1100, the virtual iron sight comprises two concentric rings (e.g., which may be circular or have another similar shape, such as a regular polygon) having fixed position relative to each other and a third ring (e.g., which may be circular or have another similar shape, such as a regular polygon) having a color and/or tint (e.g., hue, saturation, or brightness) that contrasts with the two concentric rings. The third ring generally has a visually detectable offset relative to the two concentric rings when a center of one or both headset lens(es) is/are misaligned with the gaze origin(s) (e.g., center(s) of corneal curvature) of the eye(s) of the wearer.

In optional step 1106, the wearer is prompted to adjust the headset based, at least in part, on an offset of the third ring relative to the other two existing. In certain embodiments, the prompt is visually identified by the wearer in that the third ring appears to have an offset relative to the other two (e.g., is not concentric with the other two). In certain embodiments, a prompt may further include some sort of notification, such as a further visual and/or an auditory notification. In optional step 1108, the wearer (or another, such as a physician, optometrist, or other healthcare provider) may adjust the VR headset (e.g., by one or more adjustment mechanisms) until the third ring is aligned (e.g., concentric) with the other two rings. Alignment may be determined by, for example, the adjustment causing the position of the virtual camera to be displayed entirely between the two concentric rings (e.g., where there is no portion visually detectable by the wearer as outside the two concentric rings, whether or not some portion of the third ring may be minorly occluded by one or both of the concentric rings).

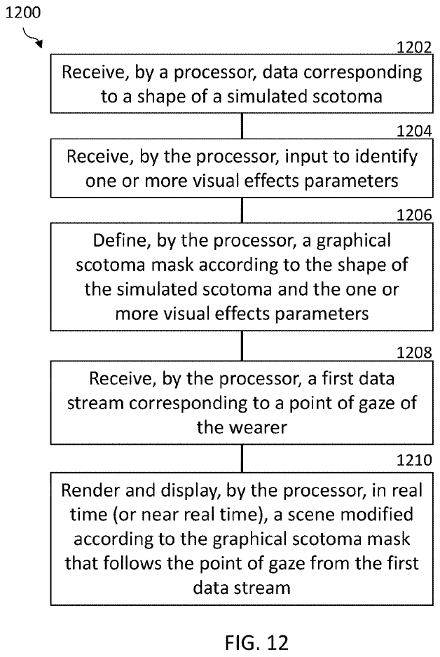

FIG. 12 illustrates an exemplary method 1200 for rendering a graphical (e.g., 2D) scotoma mask that simulates the effect of a scotoma on a visual field. In step 1202, data corresponding to a shape of a simulated scotoma is received by a processor. The data may correspond to, for example, a selected default scotoma shape, such as a disc, or a personalized scotoma shape derived from microperimetry measurement of a subject (e.g., a differential light sensitivity (DLS) map). In step 1204, input is received by the processor to identify one or more visual effects parameters corresponding to values of visual effects caused by the scotoma. For example, the input may be from a particular patient suffering from a real scotoma (e.g., who may be the patient for whom the scotoma mask is being simulated, e.g. where the scotoma mask is used to simulate disease progression). The one or more visual effects parameters may include one or more of opacity, color saturation, blur, and distortion (e.g., pincushion, barrel, or whirl). In step 1206, the graphical scotoma mask is defined by the processor according to the shape of the simulated scotoma and the one or more visual effects parameters.

In step 1208, a first data stream is received by the processor, for example from a VR headset, where the first data stream corresponds to a point of gaze of the wearer of the VR headset. In step 1210, a virtual and/or augmented scene is rendered and displayed by the processor to the wearer in his or her visual field in real time (or near real time). At least a portion of the virtual and/or augmented scene is modified according to the graphical scotoma mask. The scene may follow a point of gaze of the wearer as the point of gaze changes in real time. The virtual and/or augmented scene may be affected by the simulated scotoma using the graphical scotoma mask that follows the point of gaze of the wearer. The wearer may be an individual different from a patient, for example where the wearer desires to understand how a scotoma is affecting a particular patient's vision (e.g., where the wearer is a physician, optometrist, or other healthcare provider) or where a physician, optometrist, or other healthcare provider desires to impart an understanding to the wearer of how a scotoma may affect the wearer's vision based on a patient with a similar disease and/or disease progression.

### VR/AR/MR Device (E.g., Headset) Components

The following is a description of an illustrative VR headset system for use in various embodiments described herein. Commercially-available virtual reality (VR), augmented reality (AR), and/or mixed reality (MR) systems feature head-worn and/or face-worn hardware, as well as eye-tracking software, and may be used as components of the systems and methods described herein. As used herein, the terms “headset” or “VR headset” refer broadly to such head- or face-worn systems that implement virtual reality, augmented reality, and/or mixed reality functionality. For example, in certain embodiments, two data streams corresponding to gaze direction and gaze origin, respectively, are received from the commercial VR headset system with eye tracking capability, where the data produced has a headset reference system, rather than an eye anatomy reference system.

An example of a professional-grade VR headset system with eye tracking capability that can be used with the systems and methods described herein is the VIVE Pro Eye Office VR system, manufactured by HTC Corporation (headquartered in Xindian, New Taipei, Taiwan), as described at https://business.vive.com/us/product/vive-pro-eye-office/ and in U.S. Pat. No. 10,990,170, entitled, “Eye tracking method, electronic device, and non-transitory computer readable storage medium,” and in U.S. Pat. No. 10,705,604, entitled, “Eye tracking apparatus and light source control method thereof,” the text of each of which is incorporated herein by reference.

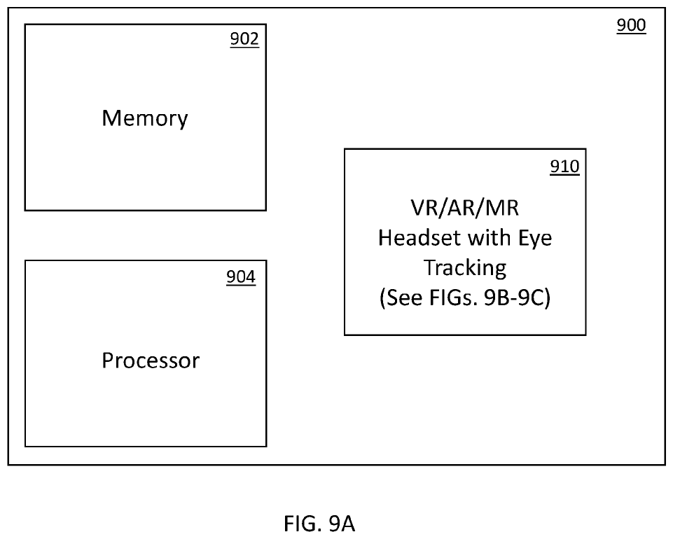

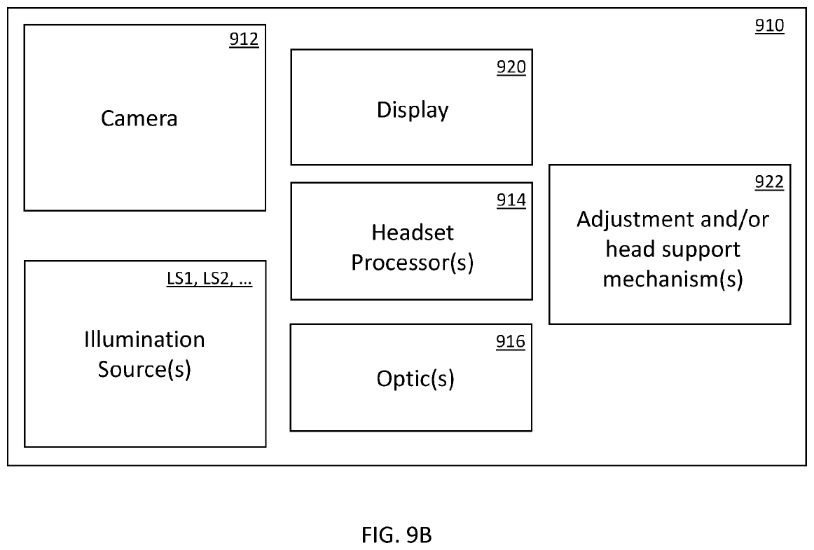

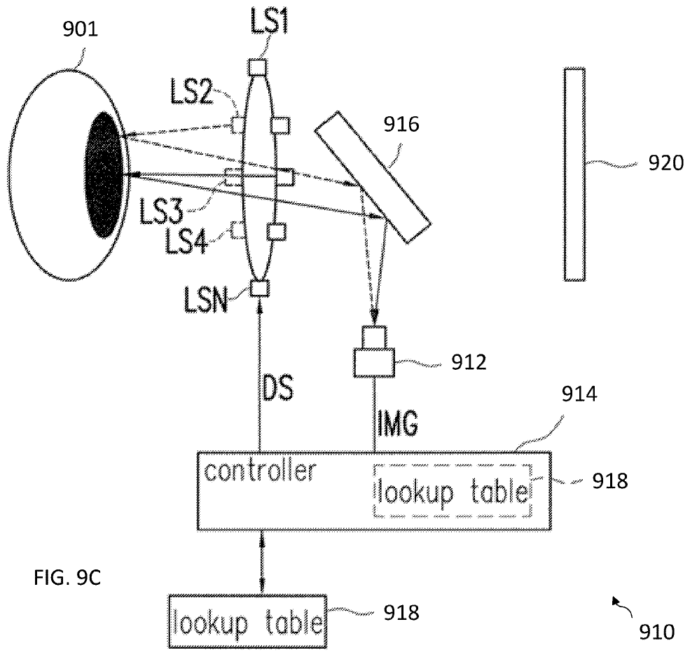

FIG. 9A shows an illustrative system 900 that can perform methods described herein. The illustrative system 900 includes a memory 902 on which instructions are stored that, when executed by processor 904, perform one or more methods described herein. Optionally, the system 900 can include a VR headset 910, for example from which a first and second data stream corresponding to a gaze direction and gaze origin are sent to, and received by, the processor 904 for use in executing instructions stored on the memory 902.

FIG. 9B shows a detailed block diagram of components that may be included in the VR headset 910, for example if the VR headset 910 is a VIVE Pro Eye Office VR system. The components may include one or more of (i) a camera 912 for tracking an eye of a subject/patient; (ii) one or more illumination sources LS1, LS2, . . . LSN for illuminating an eye of the subject-patient to provide signal to the camera 912 in order to track the eye; (iii) one or more optics 916, such as lenses, reflectors, or other light guiding components, for guiding light that has interacted with the eye (e.g., reflected from the eye) to the camera 912; (iv) a display 920 for displaying images to the subject/patient; (v) one or more headset processors 914 for processing data from camera 912, for the display 920, or from and/or for other components in the VR headset 910; and (vi) one or more adjustment and/or head support mechanisms 922 for physically adjusting (e.g., orienting and/or aligning) the VR headset on a subject/patient (e.g., relative to an eye of the subject/patient and/or for comfort during use). Generally, the VR headset 910 is a wearable apparatus that may be worn over one or both eyes of a subject/patient at a time. For example, the VIVE Pro Eye Office VR system is worn over both eyes but other VR headsets that can be used may have a “monocle” style that is worn over one eye at a time.

The display 920 may also be used to provide one or more simulated images to the subject/patient, for example based on the subject/patient's eye function as determined by a method described herein. For example, in certain embodiments, the display 920 may be used to provide the subject/patient with a simulated disease progression by way of an integrated or overlaid scotoma mask generated by the processor 904 using instructions stored on the memory 902.

The processor 904 that executes instructions may be one of the headset processors 914. The memory 902 and the processor 904 may be housed in the VR headset 910 or may be separately housed, for example in a server or other computing device that is in communication (e.g., wireless communication) with the VR headset 910. The one or more headset processors 914 may be used to send data stream(s) to the processor 902, for example wirelessly.

The VR headset 910 may also include one or more adjustment and/or head support mechanisms 922. Adjustment mechanisms 922 may include one or more mechanical mechanisms, such as knobs, straps, dials, or the like. The adjustment mechanisms 922 may be used to adjust the horizontal and/or vertical position of the headset (e.g., a component thereof, such as the display 920) relative to the head of a subject/patient. For example, the VIVE Pro Eye Office VR system includes mechanism(s) to adjust the interpupillary distance (IPD) to the particular subject/patient using the system by an “IPD knob.” IPD adjustment may involve first determining a physical IPD measurement, for example manually by the subject/patient or with assistance from a physician, optometrist, or other healthcare provider. As another example, the VIVE Pro Eye Office VR system includes a lens distance adjustment button that can be pressed to allow a subject/patient to adjust the distance of the lens further or closer to his or her face. Such an adjustment may be used to account for subject/patient anatomy or other factors, such as glasses or other sight aids. A user may be prompted to make adjustment of one or more of the adjustment mechanism(s) 922 based on methods disclosed herein that use eye tracking (e.g., in combination with a virtual iron sight (alignment aid)) to determine whether the VR headset 910 is properly aligned and/or oriented, for example illustrative method 1100.

Various head support mechanisms may be used to “mount” a VR headset on a subject/patient's head in order to secure the VR headset and/or assist in providing user comfort during use. Head support mechanisms 922 may include one or more physical structure(s) such as strap(s), mount(s), brace(s), padding, or the like. The physical structure(s) may be adjustable (e.g., a hook and loop fastener or elastic strap) or compliant (e.g., foam padding) or both (e.g., an adjustable strap with padding). For example, the VIVE Pro Eye Office VR system includes a replaceable face cushion that provides compliant support around a subject/patient's eyes for comfort as well as a head pad, adjustment dial, and center strap that collectively secure the system to a subject/patient's head with the adjustment dial being part of the head pad that sits on the back of the head and center strap that runs over the top of the head. The adjustment dial can adjust the tension of the center strap and the center strap also has a hook and loop fastener for easy mounting and dismounting from the head.

Additional details about certain illustrative adjustment and/or head support mechanisms that can be included in an embodiment of a VR headset, such as the VR headset 910 shown in FIGS. 9A-9C, are provided in the VIVE Pro Eye User guide for the VIVE Pro Eye Office VR system. Other adjustment and/or head support schemes can be used. Furthermore, in certain embodiments, a particular mechanism (e.g., structure) may serve as both an adjustment mechanism and a head support mechanism. For example, a strap may be used to secure a VR headset to a wearer and may also be used to adjust a physical position of the VR headset to the wearer's eyes.

FIG. 9C shows a schematic of how the illustrative VR headset 910 can be used to track an eye 901 of a subject/patient. Illumination sources (light sources) LS1, LS2, . . . , LSN provide light to the eye 901. Light is received by reflector 916 from the eye 901 after illumination and reflected toward the camera 912 where it is detected and the processed using a headset processor 914 that is part of a controller, which refers to lookup table 918. Optionally, the display 920 simultaneously displays image(s) to the subject/patient, for example to prompt eye movement or a particular focus of the subject/patient in order to orient or track the eye 901. One or more optics 916 (e.g., lenses) may be used to focus or otherwise guide light from the display 920 to the subject/patient. The display 920 may be considered a portion of one or more optics 916, for example a “lens” of the VR headset 910 may include the display 920 (or portion thereof).

In the illustrative VR headset 910, the illumination sources LS1, LS2, . . . , LSN project a plurality of light beams to the eye 901 on a target zone. The light reflection device 916 receives and reflects the display image IMG of the eye 901 to the camera 912. The controller with headset processor 914 is coupled to the camera 912 and the illumination sources LS1, LS2, . . . , LSN. A headset processor 914 receives the display image IMG and analyzes the contrast ratio of the display image IMG. The headset processor 914 additionally generates the command signal DS through a result of the analysis, and controls the turning on or turning off states of each of the illumination sources LS1, LS2, . . . , LSN through the command signal DS. The lookup table 918 is configured to store the relationship between the turning on/turning off states of the illumination sources LS1, LS2, . . . , LSN and the field of view information of the eyeball. The lookup table 918 may be implemented as a memory of any suitable form, which will be apparent to those of skill in the art. The lookup table 918 may be external to the controller with the headset processor 914 and coupled to the controller. Alternatively, the lookup table 918 may also be embedded in the controller with the headset processor 914. Further details of additional embodiments of how such a lookup table 918 and controller may be used to control components of the VR headset 910 to track the eye 901 of the subject/patient can be found in U.S. Pat. No. 10,705,604.

### Eye Conditions/Disorders and Functional Vision Testing

The following is a description of various eye conditions/disorders for which functional testing may be beneficial. The systems and methods described herein may be implemented in functional testing using VR headsets. For example, the systems and methods described herein utilize eye tracking implementing the technologies described herein to display static and/or changing/moving visual stimuli (targets) to the subject/patient to elicit and record patterns of eye movements relative to the stimuli, where said patterns of eye movements are correlated to a disease or condition, e.g., a retinal dysfunction. Examples of such diseases or conditions include age-related macular degeneration (AMD) and, more specifically, ‘dry’ or ‘wet’ AMD, as well as diabetic macular edema, vein or artery occlusions, and inherited retinal diseases. The functional vision testing enabled by the systems and methods described herein may also be helpful in identifying and/or monitoring cataracts, glaucoma, optic neuritis, diabetic retinopathy, and uncorrected myopia. Optical function abnormalities that can be identified and/or monitored by the systems and methods described herein may also result from (and be indicative of) multiple sclerosis, schizophrenia, or other neurological disorders.

In macular degeneration, the macula, a spot near the center of the retina, becomes damaged. As the disease progresses, the patient may experience a central scotoma. The scotoma may appear as a blurred or smudged spot, or may be a blind spot (e.g., it may progress to a gray or black spot). The size of the scotoma may increase as the disease progresses. The rest of the retina may remain undamaged, and the patient may compensate for the scotoma by shifting their gaze to see things more clearly around the central scotoma, around their direct line of sight. The patient is using peripheral vision, where rod cells are taught to perform functions that damaged cone cells once performed. In this way, the preferred retinal locus (PRL) is a retinal area that acts as a pseudofovea, compensating for a diseased fovea (area at the center of the macula). The location of the PRL (relative to retina anatomy) is important, as it can be indicative of a retinal disease or condition such as macular degeneration, and changes in the PRL over time can be tracked to monitor progression of the disease or condition. Functional tests performed using the systems and methods described herein can be used to locate the PRL relative to retina anatomy, as well as to identify abnormalities that may or may not be directly related to the PRL.

Functional tests that may be implemented include, for example, fixational stability testing, tracking stimuli moving into/out of scotoma, contrast sensitivity testing (Pelli, flicker targets), and sequential search (maze) tasks.

In an example of fixational stability testing, the subject wearing the VR headset with eye tracking capability fixates on a single target (e.g., single point) presented in the display, stationary in virtual space. The test may incorporate low contrast, high contrast, different shapes and sizes of targets, and other variations. A heat map indicative of where the subject was looking over the length of the test is produced. The system can then measure a bivariate contour ellipse area (BCEA) with results compared to known parameters in a healthy population. The PRL determination may also be a part of functional testing, for example, with changes in PRL monitored over time.

Another example of functional vision testing includes contrast sensitivity testing, for example, the “Gradiate” test as described in Mooney, S. W. J., et al. “Gradiate: A radial sweep approach to measuring detailed contrast sensitivity functions from eye movements,” Journal of Vision, 20(13): 17(2020 ) (https://doi.org/10.1167/jov.20.13.17). the text of which is incorporated herein by reference.

An improvement to contrast sensitivity functional vision testing is presented herein in which a machine learning model determines a probability that, for a given time window (in milliseconds), a subject was or was not observing an individual target. This improvement may be applied to other functional vision tests.

In developing this improvement, it is presently noted it would be beneficial during a contrast sensitivity test to have an objective classification of whether a subject was truly tracking the target or if they were looking elsewhere or otherwise moving their eyes randomly. An accurate and fast feedback loop was developed in the test where targets diminish in visibility gradually. An artificial intelligence (AI)-based visibility determination algorithm was created to determine if the subject was accurately tracking a target in time and space during the course of the contrast sensitivity functional vision test.

The AI system was developed by having users track targets in numerous trials (sample data) and then a “ground truth” was established by manually assessing video recordings after testing and annotating if the subject eye position was within an individual target(s). The ground truth dataset included hundreds of minutes of observation and annotation. The resulting AI model provided a probability that, for a given time window (in milliseconds), a subject was or was not observing an individual target (e.g., in a field of 5 targets). For functional testing, if the AI feedback indicated the subject was looking at a target, the system would alter the appearance of the target accordingly until the AI determined that the subject was no longer tracking the target. The final values of contrast and spatial frequency were recorded as the subject's visual/functional threshold for that parameter space. The subject would then move to a different target (e.g., a different sweep of the radial sweep test), and the process would repeat.

Various architectures were tested, including recombinant neural networks, long short-time memory, temporal convolutional networks, and others. Multiple threshold parameters can be adjusted to alter and improve performance. These include the time windows (e.g., increase or decrease) for the AI-determination and, in space, the tolerance for how near or far the subject's actual eye position is located from the center of the circular target.

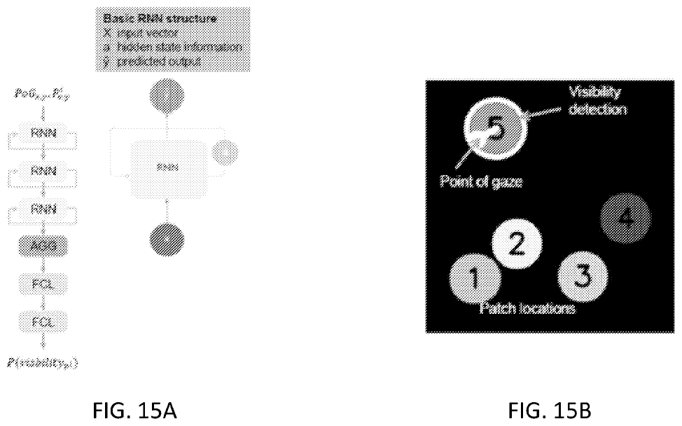

FIGS. 15A-15B are schematics of a neural network implementation for an AI-supported radial sweep functional vision test, including a visibility determination to automatically determine whether the subject is looking at a target. Shown are the example AI network architecture, and an example of the target field and visibility determination output of the AI (visibility detection). In some embodiments, a deep recurrent neural network (DRNN) architecture is used. Input to a DRNN may be 10 successive data points (including point of gaze and coordinates of one patch). A DRNN may be 3 concatenated recurrent neural networks (RNNs). FIG. 15A shows the basic structure of an RNN. A DRNN may use aggregation that takes output (e.g., 32 scalars) of a 10^{th} frame. A fully connected network (e.g., 2 layers) may be used to map hidden DRNN state to a visibility probability. The ultimate output may then be a visibility probability for one patch. A network may be trained with about two minutes of data (e.g., comparison with ground truth and backpropagation). A patch visibility prediction may be made on evaluation data (e.g., about 45 seconds) already after a very short training (e.g., about 120 seconds). FIG. 15B is a schematic representation of visibility detection with a machine learning algorithm.

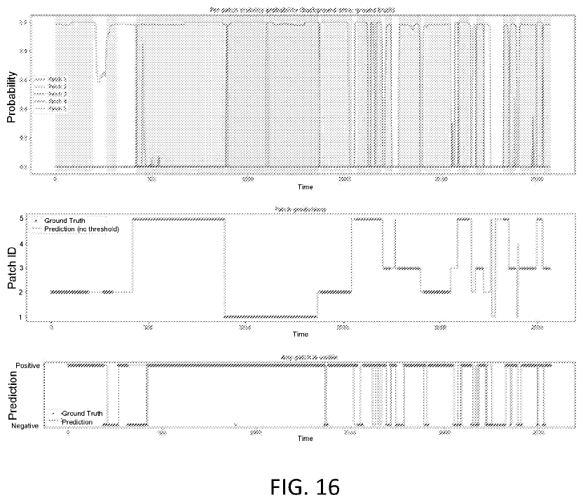

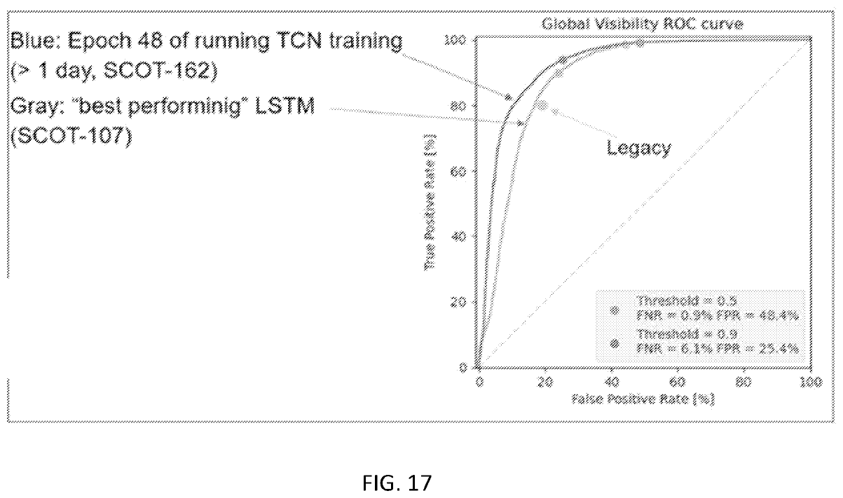

FIG. 16 are graphs indicating patch visibility probability, patch prediction, and global visibility after applying the prediction threshold in the AI-supported radial sweep functional vision test, according to an illustrative embodiment. The graphs depict output of patch visibility in a set of five targets (indicated in the legend), with time depicted on the x-axis. The “Patch prediction” graph shows “ground truth” (blue lines, manually verified) compared to prediction (yellow lines) before applying a prediction threshold, where the “Global visibility after applying prediction threshold” is a plot of ground truth vs. prediction after applying a prediction threshold. FIG. 17 is a graph depicting performance results using long short-time memory (LSTM) and temporal convolutional network (TCN) as architecture for the machine learning algorithm in the AI-supported radial sweep functional vision test, according to an illustrative embodiment.

A contrast sensitivity test was implemented using a HTC Vive Pro Eye headset. An example stillframe of the visual task used in the test is shown below. The five targets move in a random pattern around the visual field of the subject. As the subject tracks the target it decreases in contrast (making it more difficult to see). The subject is instructed to follow a target until they can no longer see it. Once all five targets are no longer visible, a new set of targets appears, and the subject repeats the task (there are a total of three ‘runs’ that account for 15 total targets).

A functional vision test, such as a contrast sensitivity and/or spatial frequency test (e.g., a radial sweep test), may be used to test eye function of a subject. A subject may be suffering from an eye condition, at risk to contract an eye condition, or be unsure of whether (s)he has an eye condition. Such an eye condition may be, for example, an age-related macular degeneration (AMD) and/or related (e.g., associated) condition. Examples of such conditions include dry age-related macular degeneration (dry AMD), wet age-related macular degeneration (wet AMD), neovascular age-related macular degeneration (AMD), and geographic atrophy. A functional vision test may be used to diagnose and/or monitor an eye condition of a subject. For example, a functional vision test may be used to generate test results that indicate presence of, severity of, and/or progression of an eye condition of a subject, such as an AMD or related (e.g., associated) condition (e.g., geographic atrophy). A virtual- and/or augmented- and/or mixed-reality device (e.g., VR head set with eye tracking capability) may be used to perform a functional vision test. In some embodiments, an eye condition is simulated with such a device, for example to demonstrate to a subject what having an eye condition would be like and/or what may occur due to progression of an eye condition that the subject may already have. In some embodiments, a scotoma may be simulated using such a device while a functional vision test is performed. A functional vision test may be used to assess quality of vision of a subject (e.g., a subject's eyesight).

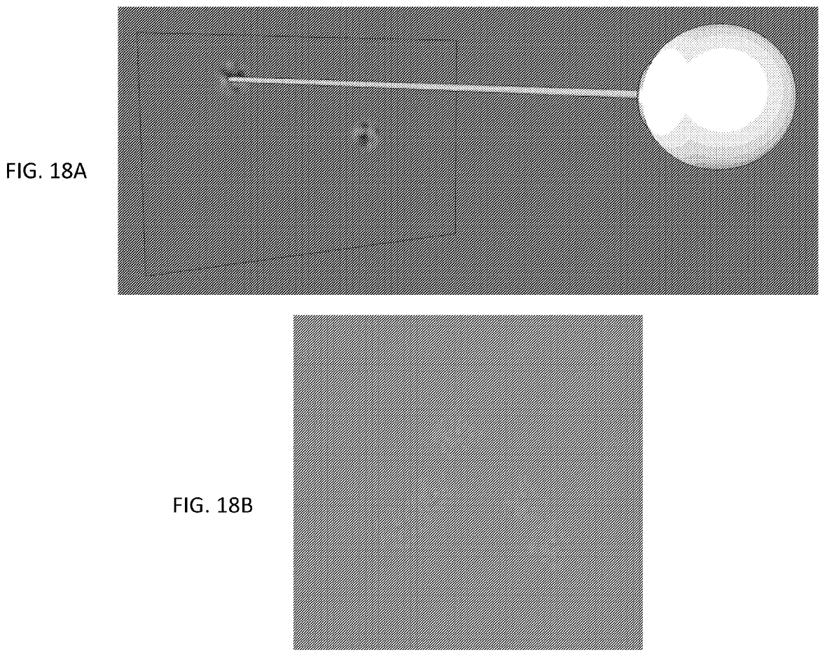

A functional eye test generally includes rendering and displaying to a subject on or more targets within a virtual and/or augmented scene in a visual field of the subject. The one or more targets may be visibility patches, for example a contrast-based visibility patch. FIGS. 18A-18B illustrate examples of contrast-based visibility patches on a chart in a virtual and/or augmented scene that may be rendered by, for example, a VR headset. One or more targets may be, two or more, three or more, four or more, or five or more targets. Target(s) may move continuously during a test. Target(s) may change contrast during a test, for example upon a determination that a subject has been tracking the target(s) for sufficient time. In some embodiments, target(s) may change direction of movement during a test. Such an abrupt change of direction may make a test more reliable at a visibility determination by reducing the likelihood of determining that a subject is actually tracking a moving target when in fact the subject was simply following a previously seen, known path. In some embodiments, an abrupt change of direction of movement of a target may be initiated based on a determination that a subject has been tracking the target less than one second (e.g., using one or more time-based parameters) (e.g., as soon as a target is seen). Adjustment (e.g., abrupt adjustment) of contrast, spatial position, spatial resolution, and/or direction of movement may be done automatically using one or more time-based parameters. Such an adjustment may make tracked target(s) lower contrast to increase difficulty, change direction of movement of tracked target(s) in an abrupt way to increase difficulty, reset all target(s) with higher contrast, and/or stop a functional vision test. For example, if a subject has not tracked any target for an appreciable period of time (e.g., at least 5 seconds), a test may be stopped or every target may have its contrast reset. The former case may occur if a subject is not engaging with a test and the latter case may occur if a subject can no longer see any target because all are too low contrast. During a test, contrast and/or spatial resolution of one or more targets may be automatically adjusted (e.g., decreased) within a virtual and/or augmented scene based on a comparison of a point of gaze to a current spatial position of the one or more targets within the virtual and/or augmented scene. For example, automatic adjustment may occur based on a determination that the point of gaze is aligned to the one or more targets for a (or at least a) predetermined threshold period of time using one or more time-based parameters.

A functional vision test may include automatically performing, in real time, a visibility determination to automatically identify if a subject is tracking one or more targets (e.g., in time and space) within a virtual and/or augmented scene. Tracking may be determined by comparing spatial location of target(s) to a point of gaze of a subject. Point of gaze may be determined with, for example, a VR headset. Point of gaze determinations may be made in real time. In some embodiments, tracking is considered to occur when a point of gaze is aligned with a target over a period of time. A point of gaze may be considered to be aligned with a target if it is coincident with a target or, in some embodiments, at least within a local proximity to a target [e.g., within an area that is slightly larger (e.g., 10% larger) than a target]. FIG. 18A is a schematic that represents an eye of a subject and the line extending from the eye towards the virtual and/or augmented scene represents a point of gaze of the eye, which in this case is aligned with (specifically in this case incident on) a target (in this case a contrast-based visibility patch).

A visibility determination may be made using one or more time-based parameters, preferably multiple time-based parameters. Use of appropriate time-based parameters may simplify the process of determining whether alignment between a point of gaze and a target (e.g., tracking of the target) is intentional, because a subject is observing and/or tracking the target, or incidental. For example, the Gradiate test described by Mooney et al. uses many parameters (more than 10 parameters), including non-time based parameters, to make visibility determinations which can make its test (i) unreliable or less reliable than it could be (for example due to complication in making visibility determination(s) using so many differing parameters). In comparison, in some embodiments, a functional vision test disclosed herein uses no more than In some embodiments, time-based parameters are used and no artificial intelligence need be used (e.g., is not used). Thus, visibility determination may be made in a different manner than described in the Gradiate test. A time-based parameter may time correspond to a time period during which the point of gaze is or is not aligned with one or more targets within a virtual and/or augmented scene. For example, in some embodiments, four time-based parameters are used. A first time-based parameter may be for tracking when a point of gaze of a subject is on a particular target within a virtual and/or augmented scene. A second time-based parameter may be for tracking when a point of gaze of a subject is off a particular target within a virtual and/or augmented scene. A third time-based parameter may be for tracking when a point of gaze of a subject is on any of a plurality of targets within a virtual and/or augmented scene. A fourth time-based parameter may be for tracking when a point of gaze of a subject is off all of a plurality of targets within a virtual and/or augmented scene.

This paragraph describes an illustrative (non-limiting) example of using a set of four time-based parameters to make a visibility determination. The parameters may have units in seconds (e.g., fractions thereof). Each target moves around and has an evidence counter associated with it (minimum value 0%, maximum value 100%). If a point of gaze of a subject is aligned with (e.g., incident on or in proximity to) a specific target, its associated evidence counter is filling up with a specific speed. A first time-based parameter governs when the evidence counter fills. The first time-based parameter may be 0.5 s to fill to 100% for tracking the specific target. That is, for 0.5 s of the point of gaze being aligned with the specific target, the evidence counter would fill from 0% to 100%. If the point of gaze is anywhere else and not on that specific target anymore, the evidence counter is decreasing with a specific speed. A second time-based parameter governs when the evidence counter empties. The second time-based parameter may be 1 s to empty to 0% for not tracking the specific target. That is, for 1 s of the point of gaze not being aligned with the specific target, the evidence counter would empty from 100% to 0%. Each evidence counter can increase while the subject is looking at the corresponding target and decrease while the subject is looking anywhere else. In general, the (visibility) evidences of all targets [e.g., all five targets (e.g., all five visibility patches)] can at any time independently be somewhere between 0% and 100% depending on where the subject's point of gaze is right now and where it has been in the near past. Thus, one counter may be increasing while other(s) are decreasing. If evidence of tracking corresponding to a specific target reaches 100%, the system assumes the target is seen by the subject (that is there is conscious recognition and the point of gaze was not only accidentally over the target). The appearance (contrast and/or spatial frequency) of that target is consequently modified to make the task more difficult. If no target is being tracked (e.g., seen anymore) (all of the local evidences are decreasing), a global evidence is then also decreasing slowly towards 0% with a certain speed. A third time-based parameter governs when the global evidence counter empties. The third time-based parameter may be 6 s to empty to 0% for not tracking any target. That is, for 6 s of the point of gaze not being aligned with any target in a virtual and/or augmented scene, the global evidence counter would empty from 100% to 0%. Similarly, if a local evidence is increasing also the global evidence increases. A fourth time-based parameter governs when the global evidence counter fills. The fourth time-based parameter may be 4 s to fill to 100% for tracking any of the targets. That is, for 4 s of the point of gaze being aligned with any target in a virtual and/or augmented scene, the evidence counter would fill from 0% to 100%. In some embodiments, if the global evidence counter is 0%, the system assumes no patch can be seen anymore and 5 new patches with high contrast are shown. Contrast and/or spatial resolution of any specific one of the target(s) (e.g., contrast-based visibility patches) may be automatically decreased when the evidence counter fills (e.g., when the subject has had his/her point of gaze aligned with the specific one for 0.5 s longer than the subject has not had his/her point of gaze aligned with the specific one). Target(s) may abruptly change direction of movement while the subject is tracking it/them, for example to enhance reliability of the visibility determination.

Time-based parameters may correspond to periods of time of, for example, milliseconds or seconds, such as, for example, a period of no more than 100 ms, no more than 250 ms, no more than 500 ms, no more than 750 ms, no more than 1 s, no more than 2 s, no more than 4 s, no more than 6 s, no more than 8 s, or no more than 10 s (e.g., each parameter corresponding to an independent period of time). For example, the foregoing example used parameters corresponding to periods of time of 500 ms, 1 s, 4 s, and 6 s.

In some embodiments, four time-based parameters account for at least 80% (e.g., at least 90%, e.g., at least 95%, e.g., at least 98%, e.g., 100%) of all meaningful variables used in a functional vision test (e.g., where a meaningful variable is a variable that has at least a 5% impact on outcome of the functional vision test). In some embodiments, a visibility determination is performed using only time-based parameters (i.e., no non-time based parameters are used to make the visibility determination) (e.g., only four time-based parameters). In some embodiments, no more than 10 total parameters (e.g., no more than 8 total parameters, no more than 6 total parameters, no more than 5 total parameters, or no more than 4 total parameters) are used to perform a visibility determination, preferably each of the parameters is a time-based parameter.

At least one time-based parameter used in a visibility determination may correspond to a subject tracking a target, for example during which a point of gaze is aligned with (e.g., incident on) the target within a virtual and/or augmented scene. At least one time-based parameter used in a visibility determination may correspond to a subject not tracking a target, for example during which a point of gaze is not aligned with (e.g., incident on) the target within a virtual and/or augmented scene. A combination of these two different types of time-based parameters may be used.

Time-based parameters may be asymmetric, for example as discussed in the previous example of a set of four time-based parameters. For example, a parameter for tracking when a point of gaze is on (e.g., aligned with) a target may refer to (e.g., correspond to) a different time length than a parameter for tracking when the point of gaze is off (e.g., not aligned with) the target (e.g., 0.5 s vs. 1 s for the corresponding parameters in the previous example). The “on” parameter may refer to a shorter time length than the “off” parameter. As another example, a parameter for tracking when a point of gaze is on (e.g., aligned with) any of a plurality of targets may refer to (e.g., correspond to) a different time length than a parameter for tracking when the point of gaze is off (e.g., not aligned with) all of the targets (e.g., 4 s vs. 6 s for the corresponding parameters in the previous example). The “on” parameter may refer to a shorter time length than the “off” parameter.

Performing a visibility determination may include determining, in real time, a point of gaze of a subject and comparing the point of gaze to a current spatial position of one or more targets within the virtual and/or augmented scene. Such a comparison may use time-based parameters. Performing a visibility determination may include determining, in real time, a point of gaze of a subject and determining whether the point of gaze is aligned with a target within the virtual and/or augmented scene. Performing a visibility determination may include determining, in real time, a point of gaze of a subject and determining whether the subject is tracking a target within the virtual and/or augmented scene, for example using at least one of the time-based parameters.

A visibility determination may signify that a subject was able to see one or more targets (e.g., was able to track one or more moving targets) (e.g., for a period of time). A radial sweep test may include making one or more visibility determinations. A visibility determination may be made using a processor in a VR headset or a processor in communication with a VR headset, for example.

Test results may be computed and/or displayed using a visibility determination. In some embodiments, test results comprise a function of contrast sensitivity (e.g., inverse of root-mean-square (RMS)) and spatial frequent (in cycles per degree, CPD) (e.g., stored as or presented in a plot). Test results from a functional vision test may be an outcome measure for an eye condition (e.g., affecting one or both eyes of the subject). A functional vision test may be a test for an eye condition such as an AMD or related (e.g., associated) condition (e.g., geographic atrophy). The test may be an outcome measure and/or a functional endpoint. A label for a packaged pharmaceutical composition or kit may include a label that includes a description and/or code that identifies a therapeutic agent that is for treatment of an eye condition, where the eye condition has been monitored and/or diagnosed by a functional vision test. In some embodiments, an eye condition of a subject is diagnosed by a method other than a functional vision test, for example by imaging (e.g., optical coherence tomography and/or fundus autofluorescence), and is subsequently monitored by a functional vision test. A label may indicate that a subject (e.g., patient) should be prescribed a therapeutic agent when progress of an eye condition is observed using test results from a functional vision test. Test results may include (e.g., be) a metric that corresponds to functional vision of the subject, such as, for example, an area under curve (AUC) metric. An AUC metric may be a “sparse” AUC metric, for example wherein one or more radial sweeps have not been performed and/or not considered. In some embodiments, a metric (e.g., an AUC metric) has at least a 90% sensitivity at 100% specificity, at least 91% sensitivity at 100% specificity, at least 92% sensitivity at 100% specificity, or at least 92.5% sensitivity at 100% specificity. In some embodiments, a metric (e.g., an AUC metric) has at least a 90% sensitivity, at least 91% sensitivity, at least 92% sensitivity, or at least 92.5% sensitivity. Test results may be calculated using a processor in a VR headset or a processor in communication with a VR headset, for example. Test results may be displayed using a processor in a VR headset or a processor in communication with a VR headset, for example.

A functional vision test may be used to diagnose and/or monitor a subject having an eye condition. A subject may be treated with a therapeutic agent (e.g., by administering a therapeutically effective amount of the therapeutic agent) based on the diagnosis and/or monitoring. For example, a subject may be determined to have a worsening severity of an eye condition that is an indication for treatment, for example administration of a therapeutically effective amount of a therapeutic agent. The eye condition may be, for example, diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., dry age-related macular degeneration (dry AMD), wet age-related macular degeneration (wet AMD), and/or neovascular age-related macular degeneration (neovascular AMD)). The diagnosing and/or monitoring of (e.g., determining of a worsening severity of) (e.g., a progressive worsening of) (e.g., a poor progression of) an eye condition may use a functional vision test disclosed herein. In some embodiments, therapeutic effectiveness of a therapeutic agent administered to a subject is determined using a functional vision test disclosed herein, for example from test results from the functional vision test. Therapeutic effectiveness may be determined for a particular subject. In some embodiments, a functional vision test may be used in conjunction with a population of subjects to determine therapeutic effectiveness of a candidate therapeutic agent and/or therapeutic intervention (e.g., in a clinical trial, e.g., to establish whether an endpoint of the trial has been met).

A therapeutic agent may be (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, or (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy. A therapeutic intervention may be laser coagulation therapy.

In some embodiments a therapeutic agent comprises a complement inhibitor complement inhibitor (e.g., a C3 inhibitor or a C5 inhibitor). In some embodiments, a complement inhibitor comprises a peptide, protein, antibody, aptamer, or small molecule that binds to a complement component or a biologically active fragment thereof (or to a complex comprising two or more complement components or biologically active fragments thereof) and inhibits its activity. In some embodiments, the complement component or biologically active fragment thereof is C3, C3a, C3b, C4, C4a, C4b, C5, C5a, C5b, C1, C1q, factor B, or factor D. In some embodiments, a complement inhibitor comprises a nucleic acid, e.g., an siRNA or antisense oligonucleotide, that inhibits expression of a complement component (e.g., C3, C5, factor B, factor D, or C1).

In some embodiments, a therapeutic agent is a gene therapy. The gene therapy may genetically modify cells in the eye so as to inhibit expression of a pathogenic gene product, correct a mutation in a gene, deliver a functional copy of a gene to cells that harbor a dysfunctional copy of such gene, or cause cells to express a beneficial nucleic acid or protein.

In some embodiments, a therapeutic agent is a cell therapy. The cells may comprise, e.g., stem cells (e.g., induced pluripotent stem cells), stem cell derived cells, retinal pigment epithelial cells, or photoreceptor cells. The cells may replace cells that have been lost to disease or support or supplement the function of remaining cells.

The following is an illustrative (non-limiting) example of functional vision testing using a visibility determination discussed previously as the example of using four specific time-based parameters. To test the usefulness of the functional vision test, scotomas of varying severity (mild or severe) were simulated while running the test as were iterations of the test without any simulated scotoma and test results were compared. Simulated mild or severe scotomas were designed from a published real patient microperimetry map. Two levels of severity (0.2 and 0.6) from a range 0 -1.0 were selected. The scotomas were the same size and shape, with the input settings adjustable with both opacity and blur, based on literature reports of vision loss in geographic atrophy (GA).

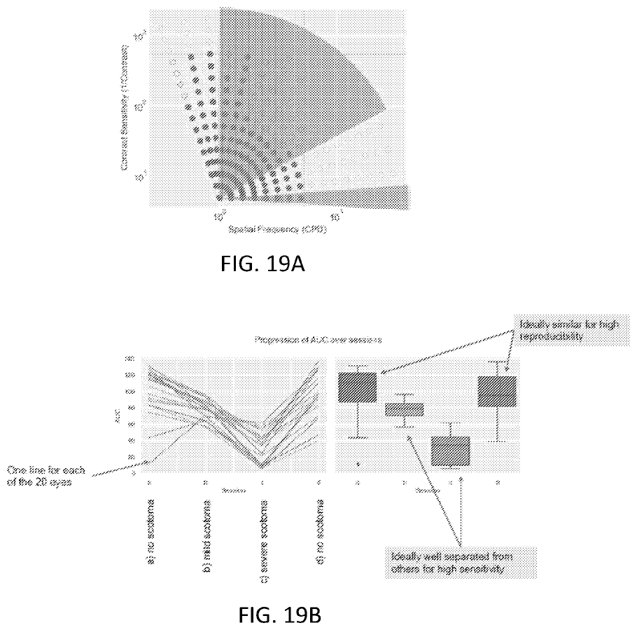

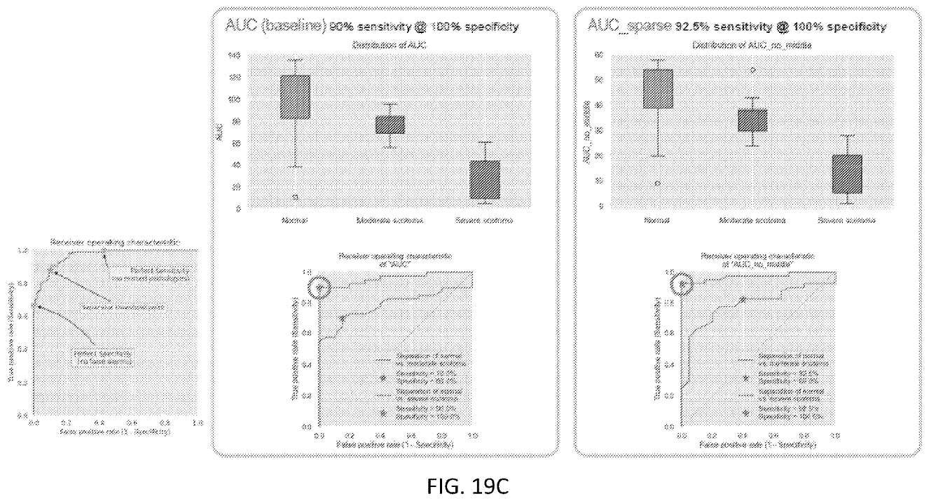

The performance of the contrast sensitivity test was assessed in a group of adult subjects using the simulated mild or severe scotomas. Some of the subjects had refractive error and used their usual corrective lenses during the test. Subjects otherwise reported no history of significant vision problems. Each subject's performance in tracking individual targets that map in the contrast-spatial frequency space was fit by a curve for each eye. The area under that curve (AUC) was determined as a metric of a subject's performance for each task, by each eye. FIGS. 19A-19C plot results of that testing. The test demonstrated reproducibility and sensitivity and was able to distinguish between a mild simulated loss and a more severe simulated loss. FIG. 19A illustrates tests results from functional vision testing. A “sparse” AUC metric was used to determine performance (see FIG. 19C) by deliberately not considering radial sweeps in sensitivity-frequency space that fall within the shaded regions. A logistic regression with L1 regularization, which revealed less sensitive sweeps that were then excluded from an AUC metric that was used, as indicated by the shaded regions in FIG. 19A. In some embodiments, an AUC metric includes fewer than 75% (e.g., fewer than 50%) of total (e.g., possible) sweeps (e.g., 6 out of 15 sweeps). Such a metric may show improved sensitivity and can enable reduced test time. However, it should be noted that some alternative radial sweep metrics will show no or limited improvement of sensitivity.

FIG. 19B shows progression of an AUC metric over different sessions for data from 20 eyes. In a first session, no simulated scotoma was used, in a second session a mild simulated scotoma was used, in a third session a severe simulated scotoma was used, and in a fourth session no simulated scotoma was used (for a second time). Comparing performance of the 20 eyes during the first and fourth sessions (both without a simulated scotoma), a high degree of reproducibility was demonstrated (shown in the right panel of FIG. 19B). Additionally, high sensitivity is shown by the well-separated box and whisker plots for the mild simulated scotoma and severe simulated scotoma. Receiver operating characteristic plots of AUC metrics were made, as shown in FIG. 19C. The panel on the left of FIG. 19C shows an illustrate ROC plot useful in understanding the ROC plots in the middle and right panels. Referring to the middle and right panels, it can be seen that a separator threshold point is higher for separation of normal vs. severe scotoma than for separation of normal vs. moderate scotoma for both AUC metrics. Moreover, the separator threshold points are higher for the sparse AUC metric than a baseline AUC metric (no radial sweeps not considered). A sensitivity at 100% specificity for the sparse AUC metric was 92.5% but only 90% for the baseline AUC metric, indicating improved sensitivity of the sparse metric.

### Decoupling Head Orientation and Virtual Objects

In some embodiments, it is important to decouple an object (e.g., test chart, such as an eye chart) and head pose of a subject, for example to enable use of low-cost, off-the-shelf VR headsets with low quality optics (e.g., Fresnel lenses). In some embodiments, a subject can align a central visual field with a specific target (e.g., contrast-based visibility patch) by rotating his or her head (and with it the headset), for example to align his or her best vision with the specific target. Allowing such reorientation can increase overall test reliability, for example because test results do not then depend on target spatial locations on a chart in a virtual and/or augmented scene. The subject can have a chance to increase perceived optical quality on a test chart (e.g., border thereof) by simply rotating his or her head (e.g., towards a border of the chart).

In some embodiments, a method includes rendering and displaying to a subject an object within a virtual and/or augmented scene in a visual field of the subject via a virtual- and/or augmented- and/or mixed-reality device (e.g., VR headset with eye tracking capability). A position of the object may remained fixed in a virtual space (e.g., fixed within the virtual and/or augmented scene) when a head of the subject changes orientation. Such an object may remain fixed when a subject reorients his or her head such that the subject can orient the object in a preferred location relative to a point of gaze of the subject. For example, a subject can orient the object to a location corresponding to alignment of best vision with an area of interest in the object (e.g., a target in a test chart). In some embodiments, a subject translating the device does not move the object relative to the subject in the virtual space. For example, in some embodiments, the subject cannot get closer or further from the object (e.g., chart) in the virtual space. In some embodiments, the object (e.g., as rendered on a head-mounted display of the VR headset) is curved (i.e., not flat), for example to account for diminished peripheral vision due to quality of one or more lenses of the device. In some embodiments, the object is a virtual test chart (e.g., eye chart), for example a test chart that includes one or more targets (e.g., moving target(s) and/or contrast-changing target(s)), for example for a functional vision test. The test chart may be curved (e.g., like a flexed piece of paper). The object may be a target rendered and displayed as part of a functional vision test. Such methods enable the use of low-cost, off-the-shelf VR headsets for vision testing, for example because they allow a subject to accommodate for inferior optics thereof (e.g., that impair a periphery of a field of view of the subject). Such methods may be used during functional vision testing.

### Uses of Methods Disclosed Herein, Including Eye Conditions and Treatment Thereof

Disclosed herein are, inter alia, methods for performing vision tests (e.g., functional vision tests) (with or without using artificial intelligence), simulating scotomas, determining a PRL, adjusting VR headsets, and displaying objects (e.g., charts) that are in a fixed position in a virtual and/or augmented scene (e.g., relative to a subject's head orientation) as well as systems for performing those methods. Any of these methods and/or systems may be used with a subject that has, or may have, an eye condition. The eye condition may be monitored and/or diagnosed with the method(s) and/or system(s). For example, determining presence of, severity of, and/or progression of an eye condition may use any method and/or system disclosed herein. The eye condition may be any eye condition mentioned herein, including, for example, diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, or age-related macular degeneration (AMD) (e.g., dry age-related macular degeneration (dry AMD), wet age-related macular degeneration (wet AMD), and/or neovascular age-related macular degeneration (neovascular AMD)). Those of ordinary skill will recognize that other eye conditions may benefit from applying a method or system disclosed herein. A system and/or method disclosed herein, such as a functional vision test (with or without use of artificial intelligence) may be used to determine therapeutic effectiveness (e.g., benefit) of a therapeutic agent and/or therapeutic intervention. Determining therapeutic effectiveness may include quantifying quality adjusted life years (QALY). Alternatively or additionally, determining cost effectiveness of a therapeutic agent and/or therapeutic intervention may be based, at least in part, on determining therapeutic effectiveness based on test results from a method disclosed herein, such as a functional vision test. Cost effectiveness may be based on quantifying QALY. A subject may be treated with a therapeutic agent and/or therapeutic intervention that has been established or confirmed to be therapeutically effective in a population of subjects using a system and/or method disclosed herein (e.g., a functional vision test). Treatment with such a therapeutic agent and/or therapeutic intervention may preserve or improve performance on a functional vision test and/or reduce rate of deterioration in performance on the functional vision test as compared to a suitable control [e.g., no treatment or sham treatment (e.g., placebo)].

Thus, a method disclosed herein, e.g., a functional vision test, can be used as a test (e.g., outcome measure or functional endpoint) for an eye condition. The test may inform and/or dictate if, how (e.g., how much), and/or when (e.g., how often) a treatment, such as a therapeutic agent and/or therapeutic intervention, for the eye condition is administered. A therapeutic agent and/or therapeutic intervention may then be administered in a therapeutically effective amount. Progression of an eye condition (e.g., progression of a severity of the eye condition) may be monitored before and/or after administering a therapeutic agent and/or therapeutic intervention. Progression may inform and/or dictate further administration of a therapeutic agent and/or therapeutic intervention. A method as disclosed herein may be used to assess therapeutic efficacy of a therapeutic agent and/or therapeutic intervention for an eye condition. Subjects that have that eye condition may then be treated with that therapeutic intervention and/or therapeutic agent.

A subject may be treated (e.g., with a therapeutic agent and/or therapeutic intervention) after having been diagnosed with an eye condition using a method and/or system disclosed herein, after an eye condition of the subject has been monitored using a method and/or system disclosed herein, while an eye condition of the subject is being monitored using a method and/or system disclosed herein, or after it has been determined that (s)he exhibits worsening severity of an eye condition using a method and/or system disclosed herein. The method and/or system may preferably be a functional vision test. A method and/or system disclosed herein may be used to diagnose and/or monitor an eye condition. A therapeutic intervention may be laser coagulation therapy.

A therapeutic agent administered to treat an eye condition after or during diagnosis and/or monitoring and/or determining of worsening severity may be, for example, (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy. In some embodiments, a therapeutic agent comprises a complement inhibitor. In some embodiments, a complement inhibitor comprises a peptide, protein, antibody, aptamer, or small molecule that binds to a complement component or a biologically active fragment thereof (or to a complex comprising two or more complement components or biologically active fragments thereof) and inhibits its activity. In some embodiments, the complement component or biologically active fragment thereof is C3, C3a, C3b, C4, C4a, C4b, C5, C5a, C5b, C1, C1q, factor B, or factor D. In some embodiments, a complement inhibitor comprises a nucleic acid, e.g., an siRNA or antisense oligonucleotide, that inhibits expression of a complement component (e.g., C3, C5, factor B, factor D, or C1). In some embodiments, a therapeutic agent is a gene therapy. The gene therapy may genetically modify cells in the eye so as to inhibit expression of a pathogenic gene product, correct a mutation in a gene, deliver a functional copy of a gene to cells that harbor a dysfunctional copy of such gene, or cause cells to express a beneficial nucleic acid or protein. In some embodiments, a therapeutic agent is a cell therapy. The cells may comprise, e.g., stem cells (e.g., induced pluripotent stem cells), stem cell derived cells, retinal pigment epithelial cells, or photoreceptor cells. The cells may replace cells that have been lost to disease or support or supplement the function of remaining cells.

### Network Environment, Computing Devices, and Software for Use in Various Embodiments

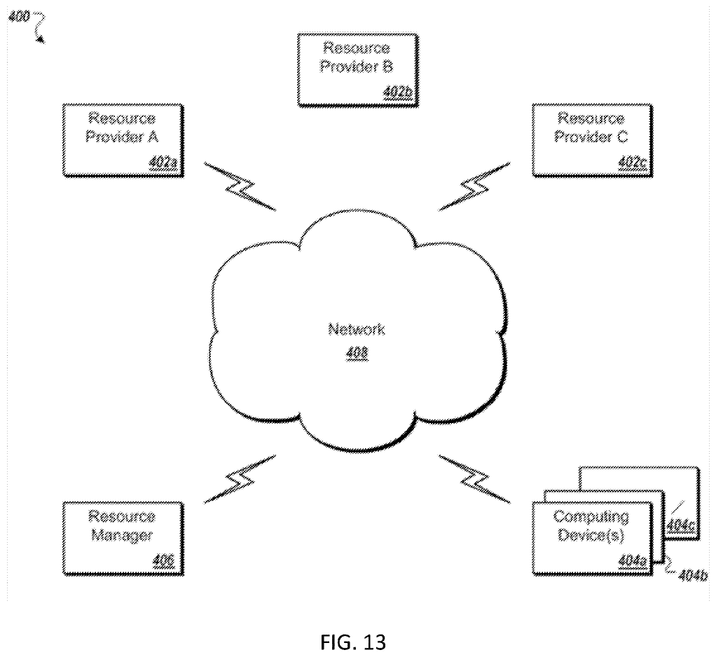

As shown in FIG. 13, an implementation of a network environment 400 for use in providing systems and methods described herein is shown and described. In brief overview, referring now to FIG. 13, a block diagram of an exemplary cloud computing environment 400 is shown and described. The cloud computing environment 400 may include one or more resource providers 402a, 402b, 402c (collectively, 402). Each resource provider 402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 402 may be connected to any other resource provider 402 in the cloud computing environment 400. In some implementations, the resource providers 402 may be connected over a computer network 408. Each resource provider 402 may be connected to one or more computing device 404a, 404b, 404c (collectively, 404), over the computer network 408.

The cloud computing environment 400 may include a resource manager 406. The resource manager 406 may be connected to the resource providers 402 and the computing devices 404 over the computer network 408. In some implementations, the resource manager 406 may facilitate the provision of computing resources by one or more resource providers 402 to one or more computing devices 404. The resource manager 406 may receive a request for a computing resource from a particular computing device 404. The resource manager 406 may identify one or more resource providers 402 capable of providing the computing resource requested by the computing device 404. The resource manager 406 may select a resource provider 402 to provide the computing resource. The resource manager 406 may facilitate a connection between the resource provider 402 and a particular computing device 404. In some implementations, the resource manager 406 may establish a connection between a particular resource provider 402 and a particular computing device 404. In some implementations, the resource manager 406 may redirect a particular computing device 404 to a particular resource provider 402 with the requested computing resource.

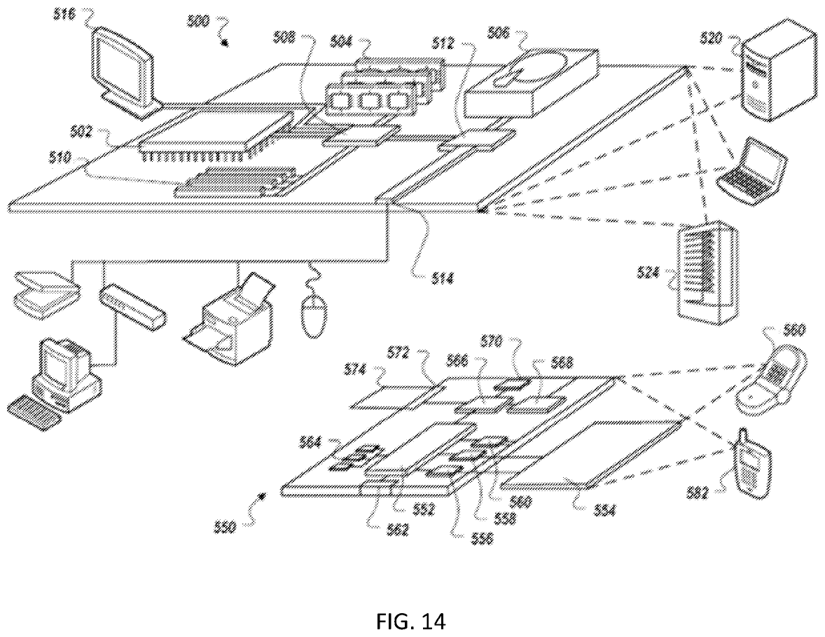

FIG. 14 shows an example of a computing device 500 and a mobile computing device 550 that can be used to implement the techniques described in this disclosure. The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk.

The storage device 506 is capable of providing mass storage for the computing device 500. In some implementations, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 502), perform one or more methods, such as those described above. The instructions can also be stored by one or more storage devices such as computer- or machine-readable mediums (for example, the memory 504, the storage device 506, or memory on the processor 502).

The high-speed interface 508 manages bandwidth-intensive operations for the computing device 500, while the low-speed interface 512 manages lower bandwidth-intensive operations. Such allocation of functions is an example only. In some implementations, the high-speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device (not shown), such as a mobile computing device 550. Each of such devices may contain one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provide as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. In some implementations, the instructions, when executed by one or more processing devices (for example, processor 552), perform one or more methods, such as those described above. The instructions can also be stored by one or more storage devices, such as one or more computer- or machine-readable mediums (for example, the memory 564, the expansion memory 574, or memory on the processor 552). In some implementations, the instructions can be received in a propagated signal, for example, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 568 using a radio-frequency. In addition, short-range communication may occur, such as using a Bluetooth®, Wi-Fi™, or other such transceiver (not shown). In addition, a GPS (Global Positioning System) receiver module 570 may provide additional navigation-and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in the figure. For example, it may be implemented as a cellular telephone 580. It may also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

The computer programs may include software that implement machine learning techniques, e.g., artificial neural networks (ANNs); e.g., convolutional neural networks (CNNs), random forest, decision trees, support vector machines, and the like, in order to determine, for a given input, one or more output values. In some embodiments, machine learning modules implementing machine learning techniques are trained, for example using curated and/or manually annotated datasets. Such training may be used to determine various parameters of machine learning algorithms implemented by a machine learning module, such as weights associated with layers in neural networks. In some embodiments, once a machine learning module is trained, e.g., to accomplish a specific task, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In some embodiments, machine learning modules may receive feedback, e.g., based on user review of accuracy, and such feedback may be used as additional training data, for example to dynamically update the machine learning module. In some embodiments, a trained machine learning module is a classification algorithm with adjustable and/or fixed (e.g., locked) parameters, e.g., a random forest classifier. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of a ANN module may be carried out via specialized hardware (e.g., via an application specific integrated circuit (ASIC), field programmable gate arrays (FPGAs), and the like).

As used herein, the terms “images”, “video”, “video stream”, and the like refer to the image data (e.g., pixel intensity values, pixel color component values (e.g., RGB, and the like), which are used to render a graphical image or sequential series of graphical images to be displayed (e.g., video). In certain embodiments, the image data received from a camera or other digital image recording device is processed as two-dimensional (2D) data. In other embodiments, the received image data is converted or mapped to three-dimensional (and/or two-and-a-half-dimensional) positions of a model. In other embodiments, the received image data is received as three-dimensional (3D) or two-and-a-half dimensional data (e.g., no conversion or mapping required).

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the software modules described herein can be separated, combined or incorporated into single or combined modules. The modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. In view of the structure, functions and apparatus of the systems and methods described here, in some implementations.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Headers are provided for the convenience of the reader-the presence and/or placement of a header is not intended to limit the scope of the subject matter described herein.

What is claimed is:

1. A method for conducting a functional vision test (e.g., a contrast sensitivity and/or spatial frequency test, e.g., a radial sweep test) on a subject using a virtual- and/or augmented- and/or mixed-reality device (e.g., VR headset with eye tracking capability), the method comprising:
rendering and displaying (e.g., on a head-mounted display of the VR headset) to the subject, by a processor of a computing device, over a course of the functional vision test, one or more targets (e.g., two or more, three or more, four or more, or five or more targets) within a virtual and/or augmented scene in a visual field of the subject;
automatically performing, by the processor, in real time, during the course of the functional vision test, a visibility determination to automatically identify if the subject is tracking the one or more targets (e.g., in time and space) within the virtual and/or augmented scene, wherein the visibility determination is based, at least in part, on time-based parameters; and
optionally, computing and/or displaying, by the processor, test results using the visibility determination.

2. The method of claim 1, wherein performing the visibility determination comprises:
determining, by the processor, in real time, a point of gaze of the subject; and
comparing, by the processor, the point of gaze to a current spatial position of the one or more targets within the virtual and/or augmented scene using the time-based parameters.

3. The method of claim 1 or claim 2, wherein performing the visibility determination comprises:
determining, by the processor, in real time, a point of gaze of the subject; and
determining, by the processor, whether the point of gaze is aligned with one of the target(s) within the virtual and/or augmented scene.

4. The method of any one of claims 1-3, wherein performing the visibility determination comprises:
determining, by the processor, in real time, a point of gaze of the subject; and
determining, by the processor, whether the subject is tracking one of the target(s) within the virtual and/or augmented scene using at least one of the time-based parameters.

5. The method of any one of claims 2-4, wherein the time-based parameters each correspond to a time period during which the point of gaze is or is not aligned with one or more of the one or more targets within the virtual and/or augmented scene.

6. The method of any one of claims 2-5, comprising automatically adjusting (e.g., decreasing) contrast and/or spatial resolution of one or more of the target(s) within the virtual and/or augmented scene based on the comparison of the point of gaze to the current spatial position of the one or more of the target(s) within the virtual and/or augmented scene (e.g., based on a determination, by the processor, that the point of gaze is aligned to the one or more of the target(s) for a (or at least a) predetermined threshold period of time using one or more of the time-based parameters).

7. The method of any one of claims 1-6, wherein there are a plurality of targets displayed within the virtual and/or augmented scene in the visual field of the subject, and wherein the time-based parameters comprises (e.g., consists of) four parameters: (i) for tracking when a (e.g., the) point of gaze of the subject is on (e.g., aligned with) a particular target of the plurality of targets within the virtual and/or augmented scene, (ii) for tracking when a (e.g., the) point of gaze of the subject is off (e.g., not aligned with) a particular target of the plurality of targets within the virtual and/or augmented scene, (iii) for tracking when a (e.g., the) point of gaze of the subject is on (e.g., aligned with) any of the plurality of targets within the virtual and/or augmented scene, and (iv) for tracking when a (e.g., the) point of gaze of the subject is off (e.g., not aligned with) all of the plurality of targets within the virtual and/or augmented scene.

8. The method of claim 7, wherein the four parameters account for at least 80% (e.g., at least 90%, e.g., at least 95%, e.g., at least 98%, e.g., 100%) of all meaningful variables used in the functional vision test (e.g., where a meaningful variable is a variable that has at least a 5% impact on the outcome of the functional vision test).

9. The method of any one of claims 1-8, wherein the time-based parameters are asymmetric [e.g., wherein parameter (i) and parameter (ii) refer to (e.g., correspond to) different time lengths and/or wherein parameter (iii) and parameter (iv) refer to (e.g., correspond to) different time lengths (e.g., wherein parameter (i) refers to a shorter time length than parameter (ii) does and/or parameter (iii) refers to a shorter time length than parameter (iv) does].

10. The method of any one of claims 1-9, wherein (i) at least one of the time-based parameters corresponds to the subject tracking one of the target(s) [e.g., during which a point of gaze is aligned with (e.g., incident on) the one of the target(s) within the virtual and/or augmented scene], (ii) at least one of the time-based parameters corresponds to the subject tracking any of the target(s) [e.g., during which a point of gaze is aligned with (e.g., incident on) any of the target(s) within the virtual and/or augmented scene], or (iii) both (i) and (ii).

11. The method of any one of claims 1-10, comprising automatically adjusting contrast, spatial position, spatial resolution, and/or direction of movement of the one or more targets (e.g., abruptly) (e.g., one or more of the one or more targets) within the virtual and/or augmented scene during the test using one or more of the time-based parameters (e.g., making a tracked one of the target(s) lower contrast to increase difficulty, changing direction of movement of a tracked one of the target(s) in an abrupt way to increase difficulty, resetting all of the target(s) with higher contrast, or stopping the test).

12. The method of claim 11, wherein adjusting the contrast, spatial position, spatial resolution, and/or direction of movement of the one or more targets comprises:
determining, by the processor, in real time, a point of gaze of the subject; and
automatically adjusting the contrast and/or spatial resolution of the one or more targets (e.g., of at least one of the one or more targets) within the virtual and/or augmented scene based at least in part on a point of gaze of the subject being aligned with the one or more targets according to at least one of the time-based parameters.

13. The method of any one of claims 1-12, wherein the visibility determination is performed using only the time-based parameters (i.e., no non-time based parameters are used to make the visibility determination).

14. The method of any one of claims 1-13, wherein no more than 10 total parameters (e.g., no more than 8 total parameters, no more than 6 total parameters, no more than 5 total parameters, or no more than 4 total parameters) are used to perform the visibility determination (e.g., and each of the parameters is a time-based parameter).

15. The method of any one of claims 1-14, wherein the one or more targets move and/or change direction of movement within the virtual and/or augmented scene during the functional vision test [e.g., regardless of whether the subject is or is not tracking the target(s) (e.g., as determined, by the processor, using a point of gaze of the subject)].

16. The method of any one of claims 1-15, wherein the one or more targets change contrast within the virtual and/or augmented scene during the functional vision test (e.g., based on determining, by the processor, in real time, that the subject is tracking the one or more targets) [e.g., changing contrast and/or spatial resolution of a target only when the subject has been tracking (e.g., continuously or intermittently) the target for a predetermined period of time)].

17. The method of any one of claims 1-16, wherein the functional vision test is a test (e.g., an outcome measure, e.g., a functional endpoint) for one or more eye conditions selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

18. The method of any one of claims 1-17, wherein the tests results are an outcome measure for an eye condition (e.g., affecting one or both eyes of the subject).

19. The method of claim 18, wherein the eye condition is selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

20. The method of any one of claims 1-19, wherein the test results indicate presence of, severity of, and/or progression of an eye condition of the subject [e.g., diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD))].

21. The method of any one of claims 1-20, wherein the test results comprise a metric that corresponds to functional vision of the subject (e.g., an area under curve (AUC) metric) (e.g., having at least a 90% sensitivity at 100% specificity, at least 91% sensitivity at 100% specificity, at least 92% sensitivity at 100% specificity, or at least 92.5% sensitivity at 100% specificity) (e.g., having at least a 90% sensitivity, at least 91% sensitivity, at least 92% sensitivity, or at least 92.5% sensitivity).

22. The method of claim 21, wherein the metric is a sparse AUC metric (e.g., wherein one or more radial sweeps have not been performed and/or not considered).

23. The method of any one of claims 1-22, wherein each of the one or more targets is a visibility patch (e.g., a contrast-based visibility patch) graphically rendered within the virtual and/or augmented scene.

24. The method of any one of claims 1-23, wherein the method is performed without use of artificial intelligence.

25. The method of any one of claims 1-24, wherein the test results comprise a function of contrast sensitivity (e.g., inverse of root-mean-square (RMS) contrast ratio) and spatial frequency (in cycles per degree, CPD) (e.g., stored as or presented in a plot).

26. The method of any one of claims 1-25, comprising simulating, by the processor, a scotoma during the functional vision test (e.g., using the method of any one of claims 84-87).

27. A packaged pharmaceutical composition or kit comprising a pharmaceutically acceptable vessel, a therapeutic agent secured or otherwise sealed within the vessel, and a label, wherein the therapeutic agent is for an eye condition [e.g., geographic atrophy and/or an age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD))], and the label comprises a description and/or code that identifies that the therapeutic agent is for treatment of the eye condition, (i) the eye condition having been diagnosed and/or monitored by a functional vision test performed according to the method of any one of claims 1-26 and/or (ii) efficacy of the therapeutic agent having been established and/or confirmed in a population of subjects (e.g., patients) by a functional vision test performed according to the method of any one of claims 1-26.

28. A method of treating a subject that has been diagnosed with an eye condition, has been or is monitored for an eye conditions, and/or has been determined to exhibit a worsening severity of an eye condition (e.g., affecting one or both eyes of the subject) using a functional vision test according to the method of any one of claims 1-26, the method comprising administering a therapeutically effective amount of a therapeutic agent (e.g., pharmaceutical compound) to the subject.

29. The method of claim 28, wherein the eye condition is selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

30. The method of claim 28 or claim 29, wherein the therapeutic agent is an antibody (e.g., a monoclonal antibody) (e.g., an anti-factor D antibody or a vascular endothelial growth factor (VEGF) inhibitor).

31. The method of claim 28 or claim 29, wherein the therapeutic agent is a vascular endothelial growth factor (VEGF) inhibitor.

32. The method of any one of claims 28-31, wherein the therapeutic agent is ranibizumab, faricimab, brolucizumab, aflibercept, or pegaptanib.

33. The method of claim 28 or claim 29, wherein the therapeutic agent comprises a vitamin supplement and/or mineral supplement (e.g., comprising vitamin C, zinc, vitamin E, copper, or beta-carotene).

34. The method of claim 28 or claim 29, wherein the therapeutic agent comprises a complement inhibitor (e.g., a C3 inhibitor or a C5 inhibitor).

35. The method of claim 34, wherein the complement inhibitor comprises a peptide, protein, antibody, or aptamer that binds to C3 and/or a biologically active fragment of C3 (e.g., C3b or C3a).

36. The method of claim 28 or claim 29, wherein the therapeutic agent is (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy.

37. A method of determining therapeutic effectiveness (e.g., benefit) of a therapeutic agent, the method comprising (i) administering a functional vision test according to the method of any one of claims 1-26 to a subject and (ii) determining therapeutic effectiveness (e.g., benefit) of a therapeutic agent to the subject based on test results from the functional vision test.

38. The method of claim 37, wherein determining the therapeutic effectiveness comprises quantifying quality adjusted life years (QALY).

39. The method of claim 37 or claim 38, comprising determining cost effectiveness of the therapeutic agent based, at least in part, on determining the therapeutic effectiveness based on test results from the functional vision test (e.g., based on quantifying QALY).

40. The method of any one of claims 37-39, wherein the therapeutic agent is (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy.

41. Use of (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy, for treatment of a subject diagnosed with and/or monitored for an eye condition using the method according to any one of claims 1-26.

42. A method of treating a subject that has been diagnosed with an eye condition, has been or is monitored for an eye conditions, and/or has been determined to exhibit a worsening severity of an eye condition (e.g., affecting one or both eyes of the subject) using a functional vision test according to the method of any one of claims 1-26, the method comprising administering a therapeutically effective therapeutic intervention (e.g., laser coagulation therapy) to the subject.

43. A method of treating a subject that has been diagnosed with an eye condition, has been or is monitored for an eye conditions, and/or has been determined to exhibit a worsening severity of an eye condition (e.g., affecting one or both eyes of the subject), the method comprising administering to the subject a therapeutically effective amount of a therapeutic agent (e.g., pharmaceutical compound), wherein the efficacy of the therapeutic agent has been established or confirmed in a population of subjects using a functional vision test according to the method of any one of claims 1-26.

44. The method of claim 43, wherein the eye condition is selected from the group consisting of: diabetic retinopathy (e.g., with or without diabetic macular retinopathy), Stargardt disease, Leber hereditary optic neuropathy (LHON), retinitis pigmentosa, glaucoma, inner nuclear layer disease, geographic atrophy, and age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (neovascular AMD)).

45. The method of claim 43 or claim 44, wherein treatment with the therapeutic agent preserves or improves performance on the functional vision test and/or reduces rate of deterioration in performance on the functional vision test as compared to a suitable control [e.g., no treatment or sham treatment (e.g., placebo)].

46. Use of a therapeutic agent for treatment of an individual diagnosed with an eye condition [e.g., geographic atrophy and/or an age-related macular degeneration (AMD) (e.g., intermediate age-related macular degeneration (intermediate AMD), dry age-related macular degeneration (dry AMD), wet age-related macular degeneration (wet AMD), and/or neovascular age-related macular degeneration (AMD)] wherein therapeutic efficacy of the therapeutic agent has been established or confirmed using a method according to any one of claims 1-26 in a population of subjects.

47. The use of claim 46, wherein the therapeutic agent comprises (i) a vitamin supplement and/or mineral supplement selected from the group consisting of vitamin C, zinc, vitamin E, copper, beta-carotene, and combinations thereof, (ii) ranibizumab, (iii) faricimab, (iv) brolucizumab, (v) aflibercept, (vi) pegaptanib, (vii) a complement inhibitor, (viii) a neuroprotective agent, (ix) an anti-inflammatory agent, (x) a free radical scavenger, (xi) an anti-apoptotic agent, (xii) an integrin modulator, (xiii) a gene therapy, or (xiv) a cell therapy.

48. A system [e.g., a virtual- and/or augmented- and/or mixed-reality device (e.g., VR headset with eye tracking capability)], comprising a processor and a memory having instructions stored thereon, the instructions executable by the processor to perform the method of any one of claims 1-26.

49. A method for conducting a vision test on a subject using a virtual- and/or augmented- and/or mixed-reality device (e.g., a VR headset with eye tracking capability), the method comprising rendering and displaying to the subject, by a processor, an object within a virtual and/or augmented scene in a visual field of the subject via the device (e.g., on a head-mounted display of the VR headset), wherein a position of the object remains fixed in a virtual space (e.g., fixed within the virtual and/or augmented scene) when a head of the subject changes orientation [e.g., such that the subject can orient the object in a preferred location relative to a point of gaze of the subject (e.g., a location corresponding to alignment of best vision with an area of interest in the object)].

50. The method of claim 49, wherein the subject translating the device does not move the object relative to the subject in the virtual space (e.g., the subject cannot get closer or further from the object in the virtual space).

51. The method of claim 49 or claim 50, wherein the object (e.g., as rendered on a head-mounted display of the VR headset) is curved (i.e., not flat) (e.g., a curved test chart) (e.g., to account for diminished peripheral vision due to quality of one or more lenses of the device).

52. The method of any one of claims 49-51, wherein the object is a virtual test chart (e.g., eye chart) (e.g., comprising one or more targets, e.g., moving target(s), e.g., for a functional vision test).

53. The method of any one of claims 49-52, wherein the VR headset is a low-cost, off-the-shelf headset.

54. A system [e.g., a virtual- and/or augmented- and/or mixed-reality device (e.g., VR headset with eye tracking capability) (e.g., a low-cost, off-the-shelf VR headset)], comprising a processor and a memory having instructions stored thereon, the instructions executable by the processor to perform the method of any one of claims 49-52.

55. A system for identifying a preferred retinal locus (PRL) relative to retina anatomy (e.g., a locus in an anatomy reference system) for one or both eyes of a subject (e.g., a patient with a macular disease such as macular degeneration, e.g., a patient with a central scotoma) (e.g., wherein the preferred retinal locus is a position on the retina other than the fovea or macula), the system comprising:
a processor of a computing device; and
a memory having instructions stored thereon, wherein the instructions, when executed by the processor, cause the processor to:
receive a first data stream (e.g., from a VR headset) corresponding to a gaze direction of an eye of the subject (e.g., said gaze direction defining a visual axis) in a headset reference system (e.g., independent of actual eye anatomy) over time;
receive a second data stream (e.g., from the VR headset) corresponding to a gaze origin at a nodal point of the eye of the subject (e.g., said nodal point corresponding to a center of corneal curvature of the eye) in a headset reference system over time, wherein the nodal point moves around a center of eye rotation as the eye rotates to change gaze direction;
identify a geometric volume (e.g., a sphere, e.g., a best fit sphere) having a reference point (e.g., a center) corresponding to the center of eye rotation and having a surface approximated by the nodal points, using data from the second data stream;
identify an anatomical reference (e.g., an optical axis of the eye) from the identified geometric volume (e.g., the sphere, e.g., the best fit sphere) (e.g., identify the optical axis of the eye as a line connecting center of the sphere and gaze origin); and
identify the preferred retinal locus (PRL) relative to retina anatomy (e.g., a locus in the anatomy reference system) using the identified anatomical reference (e.g., the optical axis of the eye) and data from the first data stream (e.g., use data from the first data stream to calculate a horizontal angle and/or vertical angle—φ, θ—between the optical axis and visual axis of the eye, and identify the PRL using the identified optical axis of the eye and the calculated horizontal angle φ and/or vertical angle θ) [e.g., and monitor the PRL (e.g., the angles φ, θ), in multiple sessions with the subject performed over time (e.g., months or years) to detect PRL changes, e.g., as disease progresses].

56. The system of claim 55, comprising a virtual- and/or augmented- and/or mixed-reality headset for producing the first data stream and the second data stream.

57. The system of claim 56, comprising an eye-tracking camera (e.g., wherein the headset comprises the eye-tracking camera).

58. The system of claim 57, comprising an illumination source (e.g., an infrared illumination source) for illuminating (the) one or both eyes of the subject (e.g., wherein the headset comprises the illumination source).

59. The system of any one of claims 56 to 58, wherein the virtual- and/or augmented- and/or mixed-reality headset comprises one or more members selected from the group consisting of: a head-mounted display, one or more lenses, one or more headset processors for producing the first data stream and/or the second data stream [e.g., wherein the processor of the computing device that executes the instructions in claim 1 is any one or more of (i) to (iv) as follows: (i) a portion or all of the one or more headset processors, (ii) distinct from the one or more headset processors, (iii) at least partially co-located with the one or more headset processors, (iv) spatially separated (remote) from the one or more headset processors, and (v) in electrical and/or data communication with the one or more headset processors], one or more mechanisms (e.g., dial, toggle, knob, or switch) for physically adjusting the position of the display in the headset, one or more mechanisms (e.g., mechanical mechanisms, e.g., knobs, straps, dials, or the like) for adjusting (e.g., manually adjusting) the horizontal and/or vertical position of the headset relative to the head of the subject, a circuit board, and a head support (e.g., strap(s), mount(s), brace(s), and/or other physical structure(s) to stabilize the headset on the head of the subject).

60. The system of any one of claims 55 to 59, wherein the instructions, when executed by the processor, cause the processor to display visual stimuli (e.g., one or more static and/or changing/moving graphical targets) to the subject to elicit and record patterns of eye movements relative to the stimuli using the identified PRL, where said recorded patterns of eye movements are correlated to a disease or condition (e.g., a retinal dysfunction).

61. The system of claim 60, wherein the instructions, when executed by the processor, cause the processor to identify said subject may have said disease or condition based at least in part on said recorded patterns of eye movements (e.g., and wherein the instructions, when executed by the processor, cause the processor to present a graphical display (e.g., an alphanumeric) indicating the subject may have said disease or condition).

62. A system for prompting adjustment (e.g., physical adjustment, e.g., manual adjustment by the wearer) of a virtual- and/or augmented- and/or mixed-reality headset position relative to a wearer's head (e.g., to improve/optimize alignment of the center of the headset lens(es) with the center of the (respective) eye(s) of the wearer), the system comprising:
a virtual- and/or augmented- and/or mixed-reality headset with one or more mechanisms (e.g., mechanical mechanisms, e.g., knobs, straps, dials, or the like) for adjusting (e.g., manually adjusting) the vertical position of the headset relative to the head of the wearer;
a processor of a computing device; and
a memory having instructions stored thereon, wherein the instructions, when executed by the processor, cause the processor to:
receive a first data stream corresponding to wearer eye position relative to the headset (e.g., the first data stream corresponding to gaze origin at a nodal point of (each of) one or both eyes of the wearer, each said nodal point corresponding to a center of corneal curvature of the (respective) eye) (e.g., the position of the center of one or both eyes of the wearer relative to the center of one or both respective headset lenses) over time, wherein a position of a virtual camera is linked to the wearer eye position relative to the headset; and
render and display a virtual iron sight in a visual field of the headset wearer in real time (or near real time) corresponding to real-time (or near real time) position of the virtual camera as the first data stream is received, said iron sight comprising two concentric rings having fixed position relative to each other and a third ring having a color and/or tint that contrasts with the two concentric rings, said third ring having a visually detectable offset relative to the two concentric rings when a center of one or both respective headset lens(es) is/are misaligned with the gaze origin(s) (center(s) of corneal curvature) of the respective eye(s) of the wearer,
wherein an offset of the third ring relative to the two concentric rings as it appears to the wearer in the visual field of the headset prompts physical adjustment (e.g., by the wearer) of the vertical position of the headset via the one or more (e.g., mechanical) mechanisms until said adjustment causes the center of one or both respective headset lens(es) to be aligned with the gaze origin(s) of the respective eye(s) of the wearer, and the resulting position of the virtual camera causes the third ring to be displayed entirely between the two concentric rings.

63. The system of claim 62, wherein the virtual- and/or augmented- and/or mixed-reality headset produces the first data stream.

64. The system of claim 63, comprising an eye-tracking camera (e.g., wherein the headset comprises the eye-tracking camera).

65. The system of claim 64, comprising an illumination source (e.g., an infrared illumination source) for illuminating (the) one or both eyes of the wearer (e.g., wherein the headset comprises the illumination source).

66. The system of any one of claims 63 to 65, wherein the virtual- and/or augmented- and/or mixed-reality headset comprises one or more members selected from the group consisting of: a head-mounted display, one or more lenses, one or more mechanisms (e.g., dial, toggle, knob, or switch) for physically adjusting the position of the display in the headset, a circuit board, and a head support (e.g., strap(s), mount(s), brace(s), and/or other physical structure(s) to stabilize the headset on the head of the subject).

67. The system of any one of claims 62 to 66, wherein the instructions, when executed by the processor, cause the processor to—following said adjustment that causes the center of one or both respective headset lens(es) to be aligned with the gaze origin(s) of the respective eye(s) of the wearer—display visual stimuli (e.g., one or more static and/or changing/moving graphical targets) to the subject to elicit and record patterns of eye movements relative to the stimuli, where said recorded patterns of eye movements are correlated to a disease or condition (e.g., a retinal dysfunction).

68. The system of claim 67, wherein the instructions, when executed by the processor, cause the processor to identify said subject may have said disease or condition based at least in part on said recorded patterns of eye movements (e.g., and wherein the instructions, when executed by the processor, cause the processor to present a graphical display (e.g., an alphanumeric) indicating the subject may have said disease or condition).

69. A system for rendering a graphical (e.g., 2D) scotoma mask that simulates the effect of a scotoma on a visual field, said scotoma suffered by a particular patient, the system comprising:
a processor of a computing device; and
a memory having instructions stored thereon, wherein the instructions, when executed by the processor, cause the processor to:
receive data corresponding to a shape of a simulated scotoma (e.g., a selected default scotoma shape such as a disc, or a personalized scotoma shape derived from microperimetry measurement of a subject, e.g., a differential light sensitivity (DLS) map);
receive input (e.g., from the particular patient suffering from a real scotoma) to identify one or more visual effects parameters corresponding to values of visual effects caused by the scotoma (e.g., said one or more parameters comprising one or more members selected from the group consisting of opacity, color saturation, blur, and distortion (e.g., pincushion, barrel, or whirl));
define the graphical scotoma mask according to the shape of the simulated scotoma and the one or more visual effects parameters;
receive a first data stream (e.g., from a VR headset) corresponding to a point of gaze of the wearer of the VR headset; and
render and display in real time (or near real time) to the wearer of the VR headset a virtual and/or augmented scene in the visual field of the wearer, at least a portion of said virtual and/or augmented scene modified according to the graphical scotoma mask, said virtual and/or augmented scene following a point of gaze of the wearer as said point of gaze changes in real time and as said virtual and/or augmented scene is affected by the simulated scotoma (e.g., wherein the wearer may be the patient or wherein the wearer may be an individual different from the patient).

70. The system of claim 69, wherein the instructions cause the processor to identify the one or more visual effects parameters from patient input by:
for a first period of time, (i) blocking vision of a healthy eye of the patient and (ii) displaying a virtual scene to the scotoma-affected eye of the patient or allowing viewing of a real field of view by the scotoma-affected eye of the patient, said patient having a one-sided scotoma;
for a second period of time, rendering and displaying (e.g., via the VR headset) the virtual scene and/or an augmented scene to only a (single) healthy eye of the patient a simulated scotoma, said virtual and/or augmented scene modified according to a graphical scotoma mask corresponding to a given shape and one or more adjustable visual effects parameters; and
updating the virtual and/or augmented scene according to feedback from the patient and rendering and displaying the updated virtual and/or augmented scene to the healthy eye of the patient in real time (or near real time), such that the patient may compare the patient's field of view in each eye and adjust the one or more visual effects parameters (and/or the scotoma shape) to match the field of view as seen by the eye with the real scotoma with the field of view as seen by the eye with the simulated scotoma.

71. The system of claim 69 or 70, comprising a virtual- and/or augmented- and/or mixed-reality headset which produces the first data stream.

72. The system of any one of claims 69 to 71, comprising an eye-tracking camera (e.g., wherein the headset comprises the eye-tracking camera).

73. The system of any one of claims 69 to 72, comprising an illumination source (e.g., an infrared illumination source) for illuminating (the) one or both eyes of the patient (e.g., wherein the headset comprises the illumination source).

74. The system of any one of claims 69 to 73, wherein the virtual- and/or augmented- and/or mixed-reality headset comprises one or more members selected from the group consisting of: a head-mounted display, one or more lenses, one or more mechanisms (e.g., dial, toggle, knob, or switch) for physically adjusting the position of the display in the headset, one or more mechanisms (e.g., mechanical mechanisms, e.g., knobs, straps, dials, or the like) for adjusting (e.g., manually adjusting) the horizontal and/or vertical position of the headset relative to the head of the subject, a circuit board, and a head support (e.g., strap(s), mount(s), brace(s), and/or other physical structure(s) to stabilize the headset on the head of the subject).

75. The system of any one of claims 69 to 74, wherein the instructions, when executed by the processor, cause the processor to display visual stimuli (e.g., one or more static and/or changing/moving graphical targets) to the subject to elicit and record patterns of eye movements relative to the stimuli, where said recorded patterns of eye movements are correlated to a disease or condition (e.g., a retinal dysfunction).

76. The system of claim 75, wherein the instructions, when executed by the processor, cause the processor to identify said wearer may have (or may have a risk of) said disease or condition based at least in part on said recorded patterns of eye movements (e.g., and wherein the instructions, when executed by the processor, cause the processor to present a graphical display (e.g., an alphanumeric) indicating the wearer may have (or may have a risk of) said disease or condition).

77. A method for identifying a preferred retinal locus (PRL) relative to retina anatomy (e.g., a locus in an anatomy reference system) for one or both eyes of a subject (e.g., a patient with a macular disease such as macular degeneration, e.g., a patient with a central scotoma) (e.g., wherein the preferred retinal locus is a position on the retina other than the fovea or macula), the method comprising:
receiving, by a processor of a computing device, a first data stream (e.g., from a VR headset) corresponding to a gaze direction of an eye of the subject (e.g., said gaze direction defining a visual axis) in a headset reference system (e.g., independent of actual eye anatomy) over time;
receiving, by the processor, a second data stream (e.g., from the VR headset) corresponding to a gaze origin at a nodal point of the eye of the subject (e.g., said nodal point corresponding to a center of corneal curvature of the eye) in a headset reference system over time, wherein the nodal point moves around a center of eye rotation as the eye rotates to change gaze direction;
identifying, by the processor, a geometric volume (e.g., a sphere, e.g., a best fit sphere) having a reference point (e.g., a center) corresponding to the center of eye rotation and having a surface approximated by the nodal points, using data from the second data stream;
identifying, by the processor, an anatomical reference (e.g., an optical axis of the eye) from the identified geometric volume (e.g., the sphere, e.g., the best fit sphere) (e.g., identify the optical axis of the eye as a line connecting center of the sphere and gaze origin); and
identifying the preferred retinal locus (PRL) relative to retina anatomy (e.g., a locus in the anatomy reference system) using the identified anatomical reference (e.g., the optical axis of the eye) and data from the first data stream (e.g., use data from the first data stream to calculate a horizontal angle and/or vertical angle—φ, θ—between the optical axis and visual axis of the eye, and identify the PRL using the identified optical axis of the eye and the calculated horizontal angle φ and/or vertical angle θ) [e.g., and monitor the PRL (e.g., the angles φ, θ), in multiple sessions with the subject performed over time (e.g., months or years) to detect PRL changes, e.g., as disease progresses].

78. The method of claim 77, comprising displaying visual stimuli (e.g., one or more static and/or changing/moving graphical targets) to the subject to elicit and record (e.g., by the processor) patterns of eye movements relative to the stimuli using the identified PRL, where said recorded patterns of eye movements are correlated to a disease or condition (e.g., a retinal dysfunction).

79. The method of claim 78, comprising identifying, by the processor, said subject as having (e.g., or, alternatively, identifying said subject as having a risk of) said disease or condition based at least in part on said recorded patterns of eye movements (e.g., and presenting, by the processor, a graphical display (e.g., an alphanumeric) indicating the subject has (e.g., or, alternatively, may have) said disease or condition).

80. A method for prompting adjustment (e.g., physical adjustment, e.g., manual adjustment by the wearer) of a virtual- and/or augmented- and/or mixed-reality headset position relative to a wearer's head (e.g., to improve/optimize alignment of the center of the headset lens(es) with the center of the (respective) eye(s) of the wearer), the method comprising:
receiving, by a processor of a computing device, a first data stream corresponding to wearer eye position relative to the headset (e.g., the first data stream corresponding to gaze origin at a nodal point of (each of) one or both eyes of the wearer, each said nodal point corresponding to a center of corneal curvature of the (respective) eye) (e.g., the position of the center of one or both eyes of the wearer relative to the center of one or both respective headset lenses) over time, wherein a position of a virtual camera is linked to the wearer eye position relative to the headset, wherein the headset comprises one or more mechanisms (e.g., mechanical mechanisms, e.g., knobs, straps, dials, or the like) for adjusting (e.g., manually adjusting) the vertical position of the headset relative to the head of the wearer; and
rendering and displaying, by the processor, a virtual iron sight in a visual field of the headset wearer in real time (or near real time) corresponding to real-time (or near real time) position of the virtual camera as the first data stream is received, said iron sight comprising two concentric rings having fixed position relative to each other and a third ring having a color and/or tint that contrasts with the two concentric rings, said third ring having a visually detectable offset relative to the two concentric rings when a center of one or both respective headset lens(es) is/are misaligned with the gaze origin(s) (center(s) of corneal curvature) of the respective eye(s) of the wearer,
wherein an offset of the third ring relative to the two concentric rings as it appears to the wearer in the visual field of the headset prompts physical adjustment (e.g., by the wearer) of the vertical position of the headset via the one or more (e.g., mechanical) mechanisms until said adjustment causes the center of one or both respective headset lens(es) to be aligned with the gaze origin(s) of the respective eye(s) of the wearer, and the resulting position of the virtual camera causes the third ring to be displayed entirely between the two concentric rings.

81. The method of claim 80, comprising checking virtual iron sign alignment during a visual function test.

82. The method of claim 80 or 81, comprising, following said adjustment that causes the center of one or both respective headset lens(es) to be aligned with the gaze origin(s) of the respective eye(s) of the wearer, displaying (e.g., by the processor) visual stimuli (e.g., one or more static and/or changing/moving graphical targets) to the wearer to elicit and record patterns of eye movements relative to the stimuli, where said recorded patterns of eye movements are correlated to a disease or condition (e.g., a retinal dysfunction).

83. The method of claim 82, comprising identifying, by the processor, that said wearer has (or may have) said disease or condition based at least in part on said recorded patterns of eye movements (e.g., and presenting a graphical display (e.g., an alphanumeric) indicating the wearer has (or may have) said disease or condition).

84. A method for rendering a graphical (e.g., 2D) scotoma mask that simulates the effect of a scotoma on a visual field, said scotoma suffered by a particular patient, the method comprising:
receiving, by the processor of a computing device, data corresponding to a shape of a simulated scotoma (e.g., a selected default scotoma shape such as a disc, or a personalized scotoma shape derived from microperimetry measurement of a subject, e.g., a differential light sensitivity (DLS) map);
receiving, by the processor, input (e.g., from the particular patient suffering from a real scotoma) to identify one or more visual effects parameters corresponding to values of visual effects caused by the scotoma (e.g., said one or more parameters comprising one or more members selected from the group consisting of opacity, color saturation, blur, and distortion (e.g., pincushion, barrel, or whirl));
defining, by the processor, the graphical scotoma mask according to the shape of the simulated scotoma and the one or more visual effects parameters;
receiving, by the processor, a first data stream (e.g., from a VR headset) corresponding to a point of gaze of the wearer of the VR headset; and
rendering and displaying, by the processor, in real time (or near real time) to the wearer of the VR headset a virtual and/or augmented scene in the visual field of the wearer, at least a portion of said virtual and/or augmented scene modified according to the graphical scotoma mask, said virtual and/or augmented scene following a point of gaze of the wearer as said point of gaze changes in real time and as said virtual and/or augmented scene is affected by the simulated scotoma (e.g., wherein the wearer may be an individual different from the patient).

85. The method of claim 84, comprising identifying, by the processor, the one or more visual effects parameters from patient input by:
for a first period of time, (i) blocking vision of a healthy eye of the patient and (ii) displaying a virtual scene to the scotoma-affected eye of the patient or allowing viewing of a real field of view by the scotoma-affected eye of the patient, said patient having a one-sided scotoma;
for a second period of time, rendering and displaying (e.g., via the VR headset) the virtual scene and/or an augmented scene to only a (single) healthy eye of the patient a simulated scotoma, said virtual and/or augmented scene modified according to a graphical scotoma mask corresponding to a given shape and one or more adjustable visual effects parameters; and
updating the virtual and/or augmented scene according to feedback from the patient and rendering and displaying the updated virtual and/or augmented scene to the healthy eye of the patient in real time (or near real time), such that the patient may compare the patient's field of view in each eye and adjust the one or more visual effects parameters (and/or the scotoma shape) to match the field of view as seen by the eye with the real scotoma with the field of view as seen by the eye with the simulated scotoma.

86. The method of claim 84 or 85, comprising displaying (e.g., by the processor) visual stimuli (e.g., one or more static and/or changing/moving graphical targets) to the wearer to elicit and record patterns of eye movements relative to the stimuli, where said recorded patterns of eye movements are correlated to a disease or condition (e.g., a retinal dysfunction).

87. The method of claim 86, comprising identifying, by the processor, said wearer may have (or may have a risk of) said disease or condition based at least in part on said recorded patterns of eye movements (e.g., and presenting a graphical display (e.g., an alphanumeric) indicating the wearer may have (or may have a risk of) said disease or condition).

88. A system for conducting a functional vision test (e.g., a contrast sensitivity and/or spatial frequency test, e.g., a radial sweep test) on a subject using a virtual- and/or augmented- and/or mixed-reality device (e.g., VR headset with eye tracking capability), the system comprising:
a processor of a computing device; and
a memory having instructions stored thereon, wherein the instructions, when executed by the processor, cause the processor to:
render and display (e.g., via the VR headset) to the subject, over a course of the functional vision test, a target (e.g., a moving target, e.g., a target that changes in spatial frequency and contrast at discrete intervals, e.g., along a plurality of sweep trajectories, e.g., where the sweeps all begin with the target at a common origin that then radiate outward along vectors in contrast sensitivity function (CSF) space until they reach the limit of function, at which point target invisibility prevents further tracking by the subject and a threshold is recorded);
automatically perform, in real time, during the course of the functional vision test, a visibility determination using a machine learning algorithm to automatically identify if the subject is accurately tracking the target in time and space; and
compute and/or display test results using the visibility determination.

89. The system of claim 88, wherein the test results comprise a plot of contrast sensitivity (e.g., inverse of root-mean-square (RMS) contrast ratio) and spatial frequency (in cycles per degree, CPD).

90. The system of claim 88 or 89, wherein the machine learning algorithm (e.g., said machine learning algorithm comprising one or more recombinant neural networks, and/or long short-time memory, and/or one or more temporal convolutional networks) has been previously trained from user trials with ground truth established by manual assessment of video recordings of the users that determined if a subject eye position was within an individual target (e.g., culled from hundreds of minutes of observation and annotation).

91. The system of any one of claims 88-90, wherein the machine learning algorithm determines, during the course of the functional vision test, a probability that, for a given time window (e.g., in milliseconds, e.g., <250 milliseconds, <100 milliseconds, <50 milliseconds, <25 milliseconds, etc.) the subject is (or is not) observing the target (e.g., an individual target in a field of a plurality of targets, e.g., 3 or more targets, e.g., 5 targets), wherein appearance of the target as presented to the subject is altered (e.g., value of contrast and/or value of spatial frequency) at least once during the course of the functional vision test upon determination the subject is (likely) observing the target (e.g., wherein the appearance of the target stops being altered when the algorithm determines the subject is no longer tracking the target, wherein final values of contrast and spatial frequency are recorded as the subject's visual/functional threshold for that parameter space, e.g., at which point a new target is presented and the process is repeated, e.g., a new sweep is conducted).

92. The system of any one of claims 88-91, wherein the instructions, when executed by the processor, cause the processor to adjust one or more threshold parameters (e.g., during the course of the functional vision test) (e.g., wherein the one or more threshold parameters comprises one or both of (i) and (ii) as follows: (i) a tolerance for how near or far the subject's actual eye position is located from the center of the target, e.g., the circular target, and (ii) a time window for the visibility determination using the machine learning algorithm.

93. A method for conducting a functional vision test (e.g., a contrast sensitivity and/or spatial frequency test, e.g., a radial sweep test) on a subject using a virtual- and/or augmented- and/or mixed-reality device (e.g., VR headset with eye tracking capability), the method comprising:
rendering and displaying (e.g., via the VR headset) to the subject, by a processor of a computing device, over a course of the functional vision test, a target (e.g., a moving target, e.g., a target that changes in spatial frequency and contrast at discrete intervals, e.g., along a plurality of sweep trajectories, e.g., where the sweeps all begin with the target at a common origin that then radiate outward along vectors in contrast sensitivity function (CSF) space until they reach the limit of function, at which point target invisibility prevents further tracking by the subject and a threshold is recorded);
automatically performing, by the processor, in real time, during the course of the functional vision test, a visibility determination using a machine learning algorithm to automatically identify if the subject is accurately tracking the target in time and space; and
computing and/or displaying, by the processor, test results using the visibility determination.

94. The method of claim 93, wherein the test results comprise a plot of contrast sensitivity (e.g., inverse of root-mean-square (RMS) contrast ratio) and spatial frequency (in cycles per degree, CPD).

95. The method of claim 93 or 94, wherein the machine learning algorithm (e.g., said machine learning algorithm comprising one or more recombinant neural networks, and/or long short-time memory, and/or one or more temporal convolutional networks) has been previously trained from user trials with ground truth established by manual assessment of video recordings of the users that determined if a subject eye position was within an individual target (e.g., culled from hundreds of minutes of observation and annotation).

96. The method of any one of claims 93-95, wherein the machine learning algorithm determines, during the course of the functional vision test, a probability that, for a given time window (e.g., in milliseconds, e.g., <250 milliseconds, <100 milliseconds, <50 milliseconds, <25 milliseconds, etc.) the subject is (or is not) observing the target (e.g., an individual target in a field of a plurality of targets, e.g., 3 or more targets, e.g., 5 targets), wherein appearance of the target as presented to the subject is altered (e.g., value of contrast and/or value of spatial frequency) at least once during the course of the functional vision test upon determination the subject is (likely) observing the target (e.g., wherein the appearance of the target stops being altered when the algorithm determines the subject is no longer tracking the target, wherein final values of contrast and spatial frequency are recorded as the subject's visual/functional threshold for that parameter space, e.g., at which point a new target is presented and the process is repeated, e.g., a new sweep is conducted).

97. The method of any one of claims 93-96, comprising adjusting, by the processor, one or more threshold parameters (e.g., during the course of the functional vision test) (e.g., wherein the one or more threshold parameters comprises one or both of (i) and (ii) as follows: (i) a tolerance for how near or far the subject's actual eye position is located from the center of the target, e.g., the circular target, and (ii) a time window for the visibility determination using the machine learning algorithm.

98. The method of any one of claims 93-97, further comprising performing steps of any one of claims 77-87.

99. The method of any one of claims 1-26, further comprising performing steps of any one of claims 77-87.

100. A method of determining therapeutic effectiveness (e.g., benefit) of a therapeutic intervention, the method comprising (i) administering a functional vision test according to the method of any one of claims 1-26 and (ii) determining therapeutic effectiveness (e.g., benefit) of a therapeutic intervention (e.g., laser coagulation therapy) to a subject based on test results from the functional vision test.
